(19) United States
(12) Patent Application Publication
(10) Pub. No.: US 2021/0318338 A1
(43) Pub. Date: Oct. 14, 2021

(54) METHOD FOR THE DETERMINATION OF PROTEIN S LEVELS

(71) Applicant: THROMBOSIS AND COAGULATION AB, Malmo (SE)

(72) Inventor: Björn DAHLBÄCK, Malmö (SE)

(21) Appl. No.: 17/282,289

(22) PCT Filed: Oct. 3, 2019
(86) PCT No.: PCT/EP2019/076801

§ 371 Date: Apr. 1, 2021

(30) Foreign Application Priority Data
Oct. 4, 2018 (SE) 1851200-4

(51) Int. Cl. G01N 33/86; G01N 21/64; G01N 21/78

(52) U.S. Cl. CPC G01N 33/86; G01N 2800/226; G01N 21/78; G01N 21/6486

(57) ABSTRACT

The present invention provides an in vitro method for the assessment of functional protein S levels in a sample. The present invention also provides kits for use in the determination of functional protein S levels in a sample. Also provided is a method of treatment based on the determination of functional protein S levels, followed by administration of a therapeutic agent.

### FIELD OF INVENTION

The present invention relates to an in vitro assay for the determination of protein S levels in a sample, in particular for determination of functional protein S levels.

### BACKGROUND

Protein S is a vitamin K-dependent plasma protein with multiple functions[1, 2]. In human plasma it circulates in two forms, as free protein (≈30%) and as complex (≈70%) with the complement regulator C4b-binding protein (C4BP)[1, 2]. The free protein S serves as cofactor to two different anticoagulant proteins, activated protein C (APC) and tissue factor pathway inhibitor alpha (TFPIα)[3-5]. The physiological importance of protein S as anticoagulant is demonstrated by the increased risk of venous thrombosis that affects individuals with heterozygous protein S deficiency[6]. A dramatic hypercoagulable state has been described in rare cases of infants with homozygous protein S deficiency. Protein S knockout mice further demonstrate the crucial importance of protein S as the knockout results in embryonic lethality[7, 8]. Interestingly, combining protein S knockout with hemophilia A (FVIII-knockout) or B (FIX-knockout) resulted in mice with no overt thrombotic or bleeding phenotype demonstrating that the lethality of protein S knockout is due to uncontrolled hypercoagulation[9, 10].

APC together with protein S regulates the activity of activated factor V (FVa) and activated factor VIII (FVIIIa), which are cofactors to the enzymes factor Xa (FXa) and factor IXa (FIXa), respectively[11, 12]. On the surface of negatively charged phospholipid, FXa and FVa form the prothrombinase complex activating prothrombin to thrombin, whereas FIXa and FVIIIa create the tenase complex that activates FX to FXa. The tenase and prothrombinase complexes are key components in the propagation phase of coagulation and APC together with protein S are important anticoagulant proteins that regulate the activity of this phase.

Protein S has also been identified to serve as cofactor to TFPIα in the regulation of FXa activity[5, 13-16]. TFPIα is a crucial regulator of the extrinsic pathway of coagulation as it inhibits the tissue factor—factor VIIa (TF/FVIIa) complex that is formed upon vascular damage when TF is exposed to blood where both FVII and FVIIa (activated form of FVII) are present[17-19]. TF/FVIIa activates both FIX and FX, thus initiating a coagulation reaction. TFPIα binds and inhibits freshly activated FXa and subsequently the TFPIα/FXa complex binds and inhibits TF/FVIIa. In the presence of negatively charged phospholipid, the rate of inhibition of FXa by TFPIα is enhanced by the presence of protein S. TFPIα is a Kunitz type protease inhibitor containing three Kunitz domains, the first binds and inhibits FVIIa, the second binds and inhibits FXa and the third binds protein S[17-19].

Recently a splice variant of coagulation factor V (FV) has been identified in which 702 amino acid residues are deleted (residues 756-1458) from the large centrally located activation domain, the B domain[20]. The B domain is cleaved off during the activation of FV to FVa by thrombin- or FXa-mediated cleavages at positions 709, 1018 and 1545 and the FV-Short splice variant is therefore fully active as procoagulant after cleavage by thrombin or FXa. However, importantly the truncation of the B domain results in the exposure of a negatively charged high affinity-binding site for TFPIα that is located in the remaining C-terminal part (residues 1458-1545) of the B domain. In the TFPIα molecule, the binding site for FV-Short is located in the highly positively charged C-terminal extension that follows after the third Kunitz domain[21-24]. The TFPIα (around 0.2 nM) that is present in plasma circulates either in a high affinity complex with FV-Short (Kd<1 nM), which is present in plasma at sub nM levels, or in a low affinity (Kd>10 nM) interaction with full length FV, which is present at around 20 nM. The binding of TFPIα to FV-Short and to full length FV is important to keep TFPIα in the circulation as it otherwise would be lost in the urine due to its relatively low molecular weight (40 kDa)[20].

It has recently been shown that the interaction between TFPIα and FV-Short is not only important for keeping TFPIα in the circulation but that also affects the function of TFPIα as inhibitor of FXa[3, 25]. FV-Short in itself only weakly stimulates the activity of TFPIα but it strongly supports the TFPIα-cofactor activity of protein S. The results suggest that FV-Short and protein S act as synergistic TFPIα cofactors. In a model system with purified components, the presence of FV-Short (just a few nM) and negatively charged phospholipid vesicles, protein S is highly efficient and just a few nM yields maximum TFPIα cofactor activity[25]. In contrast, in the absence of FV-Short, not even up to 100 nM protein S yields equally efficient TFPIα cofactor activity.

Inherited or acquired protein S deficiency is a risk factor for venous thrombosis and analysis of protein S is part of the laboratory evaluation of patients with venous thrombo-embolic disease (VTE)[2]. Antibody-based assays and tests for the APC-cofactor function of protein S are commercially available and with such assays, a few percent of patients with VTE are identified as being protein S deficient. To identify decreased protein, assays for free protein S have been demonstrated to have highest predictive value[26].

There remains a need for the identification of new and improved methods for determining protein S levels, in particular for determining levels of functional protein S.

### SUMMARY OF INVENTION

Accordingly, the present invention seeks to mitigate, alleviate or eliminate one or more of the above-identified deficiencies in the art and disadvantages singly or in any combination and solves at least the above-mentioned problems by providing methods according to the appended patent claims.

A first aspect of the present invention provides an in vitro method for the determination of functional protein S levels in a sample, wherein the method comprises the steps of:
(a) contacting a sample obtained from a subject with TFPIα and one or more of: FV-short; or an FV-short variant; or a functionally-equivalent FV-variant;
(b) contacting the sample with FXa; and
(c) measuring the level of FXa activity in the sample
wherein the level of FXa activity is indicative of the level of functional protein S in the sample.

In one embodiment, the method consists of steps (a) to (c) listed above.

The invention also provides an in vitro method for the determination of free (or non-C4BP complexed) protein S levels in a sample, wherein the method comprises the steps of:
(a) contacting a sample obtained from a subject with TFPIα and one or more of: FV-short; or an FV-short variant; or a functionally-equivalent FV-variant;
(b) contacting the sample with FXa; and
(c) measuring the level of FXa activity in the sample
wherein the level of FXa activity is indicative of the level of free (or non-C4BP complexed) protein S in the sample.

By “FV” we mean coagulation factor V, which includes at least partially-activated forms of FV (FVa), providing the FV is activated such that the TFPIα synergistic cofactor activity is retained. The TFPIα synergistic cofactor activity will be retained in partially activated forms where the C-terminus of the B domain is exposed. In fully activated FVa the whole B domain is cleaved off and therefore FVa has no interaction with TFPIα and no cofactor activity. The sequence of human FV is given below in SEQ ID NO: 1:

SEQ ID NO: 1 is the full-length sequence of mature circulating single chain FV. The section in italics at the start of the sequence is the part corresponding to the heavy chain (residues 1-709) and the section in normal font (not bold, italic or underlined) at the end of the sequence is the light chain (residues 1546-2196). The section in bold between these is the B domain (residues 710-1545).

SEQ ID NO: 1
AQLRQFYVAAQGISWSYRPEPTNSSLNLSVTSFKKIVYREYEPYFKKEKP
QSTISGLLGPTLYAEVGDIIKVHFKNKADKPLSIHPQGIRYSKLSEGASY
LDHTFPAEKMDDAVAPGREYTYEWSISEDSGPTHDDPPCLTHIYYSHENL
IEDFNSGLIGPLLICKKGTLTEGGTQKTFDKQIVLLFAVFDESKSWSQSS
SLMYTVNGYVNGTMPDITVCAHDHISWHLLGMSSGPELFSIHFNGQVLEQ
NHHKVSAITLVSATSTTANMTVGPEGKWIISSLTPKHLQAGMQAYIDIKN
CPKKTRNLKKITREQRRHMKRWEYFIAAEEVIWDYAPVIPANMDKKYRSQ
HLDNFSNQIGKHYKKVMYTQYEDESFTKHTVNPNMKEDGILGPIIRAQVR
DTLKIVFKNMASRPYSIYPHGVTFSPYEDEVNSSFTSGRNNTMIRAVQPG
ETYTYKWNILEFDEPTENDAQCLTRPYYSDVDIMRDIASGLIGLLLICKS
RSLDRRGIQRAADIEQQAVFAVFDENKSWYLEDNINKFCENPDEVKRDDP
KFYESNIMSTINGYVPESITTLGFCFDDTVQWHFCSVGTQNEILTIHFTG
HSFIYGKRHEDTLTLFPMRGESVTVTMDNVGTWMLTSMNSSPRSKKLRLK
FRDVKCIPDDDEDSYEIFEPPESTVMATRKMHDRLEPEDEESDADYDYQN
RLAAALGIRSFRNSSLNQEEEEFNLTALALENGTEFVSSNTDIIVGSNYS
SPSNISKFTVNNLAEPQKAPSHQQATTAGSPLRHLIGKNSVLNSSTAEHS
SPYSEDPIEDPLQPDVTGIRLLSLGAGEFKSQEHAKHKGPKVERDQAAKH
RFSWMKLLAHKVGRHLSQDTGSPSGMRPWEDLPSQDTGSPSRMRPWKDPP
SDLLLLKQSNSSKILVGRWHLASEKGSYEIIQDTDEDTAVNNWLISPQNA
SRAWGESTPLANKPGKQSGHPKFPRVRHKSLQVRQDGGKSRLKKSQFLIK
TRKKKKEKHTHHAPLSPRTFHPLRSEAYNTFSERRLKHSLVLHKSNETSL
PTDLNQTLPSMDFGWIASLPDHNQNSSNDTGQASCPPGLYQTVPPEEHYQ
TFPIQDPDQMHSTSDPSHRSSSPELSEMLEYDRSHKSFPTDISQMSPSSE
HEVWQTVISPDLSQVTLSPELSQTNLSPDLSHTTLSPELIQRNLSPALGQ
MPISPDLSHTTLSPDLSHTTLSLDLSQTNLSPELSQTNLSPALGQMPLSP
DLSHTTLSLDFSQTNLSPELSHMTLSPELSQTNLSPALGQMPISPDLSHT
TLSLDFSQTNLSPELSQTNLSPALGQMPLSPDPSHTTLSLDLSQTNLSPE
LSQTNLSPDLSEMPLFADLSQIPLTPDLDQMTLSPDLGETDLSPNFGQMS
LSPDLSQVTLSPDISDTTLLPDLSQISPPPDLDQIFYPSESSQSLLLQEF
NESFPYPDLGQMPSPSSPTLNDTFLSKEFNPLVIVGLSKDGTDYIEIIPK
EEVQSSEDDYAEIDYVPYDDPYKTDVRTNINSSRDPDNIAAWYLRSNNGN
RRNYYIAAEEISWDYSEFVQRETDIEDSDDIPEDTTYKKVVFRKYLDSTF
TKRDPRGEYEEHLGILGPIIRAEVDDVIQVRFKNLASRPYSLHAHGLSYE
KSSEGKTYEDDSPEWFKEDNAVQPNSSYTYVWHATERSGPESPGSACRAW
AYYSAVNPEKDIHSGLIGPLLICQKGILHKDSNMPMDMREFVLLFMTFDE
KKSWYYEKKSRSSWRLTSSEMKKSHEFHAINGMIYSLPGLKMYEQEWVRL
HLLNIGGSQDIHVVHFHGQTLLENGNKQHQLGVWPLLPGSFKTLEMKASK
PGWWLLNTEVGENQRAGMQTPFLIMDRDCRMPMGLSTGIISDSQIKASEF
LGYWEPRLARLNNGGSYNAWSVEKLAAEFASKPWIQVDMQKEVIITGIQT
QGAKHYLKSCYTTEFYVAYSSNQINWQIFKGNSTRNVMYFNGNSDASTIK
ENQFDPPIVARYIRISPTRAYNRPTLRLELQGCEVNGCSTPLGMENGKIE
NKQITASSFKKSWWGDYWEPFRARLNAQGRVNAWQAKANNNKQWLEIDLL
KIKKITAIITQGCKSLSSEMYVKSYTIHYSEQGVEWKPYRLKSSMVDKIF
EGNTNTKGHVKNFFNPPIISRFIRVIPKTWNQSIALRLELFGCDIY

By “FV-short” (also “FV-756-1458”), we mean an alternative splice variant of FV, resulting in the in-frame deletion of 702 amino acid residues from the large activation domain (B domain) of FV—between residues 756-1458 [20]. This results in the exposure of an acidic region in the remaining C-terminal part of the B domain, which constitutes a high affinity-binding site for TFPIα [23].

An exemplary amino acid sequence of FV-Short is given below as SEQ ID NO: 2. FV-short has a deletion between amino acids 756-1458 of 702 amino acids, compared to FV [20]. The section in italics at the start of the sequence is the part corresponding to the heavy chain (residues 1-709) and the section in normal font (not bold, italic or underlined) at the end of the sequence is the light chain (residues 1546-2196). The section between these (beginning and ending with the sequences in bold) is what is left of the B domain after the deletion. The bold and underlined section corresponds to positions 710-755, whereas the following bold and not underlined part represents 1458-1545 part of the full length FV sequence.

SEQ ID NO: 2
AQLRQFYVAAQGISWSYRPEPTNSSLNLSVTSFKKIVYREYEPYFKKEKP
QSTISGLLGPTLYAEVGDIIKVHFKNKADKPLSIHPQGIRYSKLSEGASY
LDHTFPAEKMDDAVAPGREYTYEWSISEDSGPTHDDPPCLTHIYYSHENL
IEDFNSGLIGPLLICKKGTLTEGGTQKTFDKQIVLLFAVFDESKSWSQSS
SLMYTVNGYVNGTMPDITVCAHDHISWHLLGMSSGPELFSIHFNGQVLEQ
NHHKVSAITLVSATSTTANMTVGPEGKWIISSLTPKHLQAGMQAYIDIKN
CPKKTRNLKKITREQRRHMKRWEYFIAAEEVIWDYAPVIPANMDKKYRSQ
HLDNFSNQIGKHYKKVMYTQYEDESFTKHTVNPNMKEDGILGPIIRAQVR
DTLKIVFKNMASRPYSIYPHGVTFSPYEDEVNSSFTSGRNNTMIRAVQPG
ETYTYKWNILEFDEPTENDAQCLTRPYYSDVDIMRDIASGLIGLLLICKS
RSLDRRGIQRAADIEQQAVFAVFDENKSWYLEDNINKFCENPDEVKRDDP
KFYESNIMSTINGYVPESITTLGFCFDDTVQWHFCSVGTQNEILTIHFTG
HSFIYGKRHEDTLTLFPMRGESVTVTMDNVGTWMLTSMNSSPRSKKLRLK
FRDVKCIPDDDEDSYEIFEPPESTVMATRKMHDRLEPEDEESDADYDYQN
RLAAALGIRSFRNSSLNQEEEEFNLTALALENGTEFVSSNTDIIVGSNYS
SPSNINLGQMPSPSSPTLNDTFLSKEFNPLVIVGLSKDGTDYIEIIPKEE
VQSSEDDYAEIDYVPYDDPYKTDVRTNINSSRDPDNIAAWYLRSNNGNRR
NYYIAAEEISWDYSEFVQRETDIEDSDDIPEDTTYKKVVFRKYLDSTFTK
RDPRGEYEEHLGILGPIIRAEVDDVIQVRFKNLASRPYSLHAHGLSYEKS
SEGKTYEDDSPEWFKEDNAVQPNSSYTYVWHATERSGPESPGSACRAWAY
YSAVNPEKDIHSGLIGPLLICQKGILHKDSNMPMDMREFVLLFMTFDEKK
SWYYEKKSRSSWRLTSSEMKKSHEFHAINGMIYSLPGLKMYEQEWVRLHL
LNIGGSQDIHVVHFHGQTLLENGNKQHQLGVWPLLPGSFKTLEMKASKPG
WWLLNTEVGENQRAGMQTPFLIMDRDCRMPMGLSTGIISDSQIKASEFLG
YWEPRLARLNNGGSYNAWSVEKLAAEFASKPWIQVDMQKEVIITGIQTQG
AKHYLKSCYTTEFYVAYSSNQINWQIFKGNSTRNVMYFNGNSDASTIKEN
QFDPPIVARYIRISPTRAYNRPTLRLELQGCEVNGCSTPLGMENGKIENK
QITASSFKKSWWGDYWEPFRARLNAQGRVNAWQAKANNNKQWLEIDLLKI
KKITAIITQGCKSLSSEMYVKSYTIHYSEQGVEWKPYRLKSSMVDKIFEG
NTNTKGHVKNFFNPPIISRFIRVIPKTWNQSIALRLELFGCDIY

Thus, in one embodiment of the invention, the FV-short of the method has the sequence of SEQ ID NO: 2 above.

Also described herein are FV-short variants. One FV-Short variant is FV 810-1491 (SEQ ID: No 3), which despite having retained the acidic region in the B domain that binds TFPIα lacks TFPIα cofactor activity together with protein S. This mutant was originally described in another context in a publication by Toso and Camire in 2004 [29].

The section in italics at the start of the sequence is the part corresponding to the heavy chain (residues 1-709) and the section in normal font (not bold, italic or underlined) at the end of the sequence is the light chain (residues 1546-2196). The section between these (beginning and ending with the sequences in bold) is what is left of the B domain after the deletion. The bold and underlined section corresponds to positions 710-810, whereas the following bold and not underlined part represents 1491-1545 part of the full length FV sequence.

SEQ ID No: 3
AQLRQFYVAAQGISWSYRPEPTNSSLNLSVTSFKKIVYREYEPYFKKEKP
QSTISGLLGPTLYAEVGDIIKVHFKNKADKPLSIHPQGIRYSKLSEGASY
LDHTFPAEKMDDAVAPGREYTYEWSISEDSGPTHDDPPCLTHIYYSHENL
IEDFNSGLIGPLLICKKGTLTEGGTQKTFDKQIVLLFAVFDESKSWSQSS
SLMYTVNGYVNGTMPDITVCAHDHISWHLLGMSSGPELFSIHFNGQVLEQ
NHHKVSAITLVSATSTTANMTVGPEGKWIISSLTPKHLQAGMQAYIDIKN
CPKKTRNLKKITREQRRHMKRWEYFIAAEEVIWDYAPVIPANMDKKYRSQ
HLDNFSNQIGKHYKKVMYTQYEDESFTKHTVNPNMKEDGILGPIIRAQVR
DTLKIVFKNMASRPYSIYPHGVTFSPYEDEVNSSFTSGRNNTMIRAVQPG
ETYTYKWNILEFDEPTENDAQCLTRPYYSDVDIMRDIASGLIGLLLICKS
RSLDRRGIQRAADIEQQAVFAVFDENKSWYLEDNINKFCENPDEVKRDDP
KFYESNIMSTINGYVPESITTLGFCFDDTVQWHFCSVGTQNEILTIHFTG
HSFIYGKRHEDTLTLFPMRGESVTVTMDNVGTWMLTSMNSSPRSKKLRLK
FRDVKCIPDDDEDSYEIFEPPESTVMATRKMHDRLEPEDEESDADYDYQN
RLAAALGIRSFRNSSLNQEEEEFNLTALALENGTEFVSSNTDIIVGSNYS
SPSNISKFTVNNLAEPQKAPSHQQATTAGSPLRHLIGKNSVLNSSTAEHS
SPYSEDPIEDTDYIEIIPKEEVQSSEDDYAEIDYVPYDDPYKTDVRTNIN
SSRDPDNIAAWNYLRSNNGNRRNYYIAAEEISWDYSEFVQRETDIEDSDD
IPEDTTYKKVVFRKYLDSTFTKRDPRGEYEEHLGILGPIIRAEVDDVIQV
RFKNLASRPYSLHAHGLSYEKSSEGKTYEDDSPEWFKEDNAVQPNSSYTY
VWHATERSGPESPGSACRAWAYYSAVNPEKDIHSGLIGPLLICQKGILHK
DSNMPVDMREFVLLFMTFDEKKSWYYEKKSRSSWRLTSSEMKKSHEFHAI
NGMIYSLPGLKMYEQEWVRLHLLNIGGSQDIHVVHFHGQTLLENGNKQHQ
LGVVVPLLPGSFKTLEMKASKPGWWLLNTEVGENQRAGMQTPFLIMDRDC
RMPMGLSTGIISDSQIKASEFLGYWEPRLARLNNGGSYNAWSVEKLAAEF
ASKPWIQVDMQKEVIITGIQTQGAKHYLKSCYTTEFYVAYSSNQINWQIF
KGNSTRNVMYFNGNSDASTIKENQFDPPIVARYIRISPTRAYNRPTLRLE
LQGCEVNGCSTPLGMENGKIENKQITASSFKKSWWGDYVVEPFRARLNAQ
GRVNAWQAKANNNKQWLEIDLLKIKKITAIITQGCKSLSSEMYVKSYTIH
YSEQGVEWKPYRLKSSMVDKIFEGNTNTKGHVKNFFNPPIISRFIRVIPK
TWNQSITLRLELFGCDIY

Another FV-Short variant which has increased TFPIα synergistic cofactor activity with protein S as compared to FV-Short is FV 709-1476 (SEQ ID No: 4) which originally was described in another context by Marquette et al in 1995 [28].

The section in italics at the start of the sequence is the part corresponding to the heavy chain (residues 1-709) and the section in normal font (not bold, italic or underlined) at the end of the sequence is the light chain (residues 1546-2196). The section between these (beginning and ending with the sequences in bold) is what is left of the B domain after the deletion (represents 1476-1545 part of the full length FV sequence). In this construct, Ile 708 is mutated to Thr and Leu 1544 is mutated to Thr. This mutant also has an introduced MluI restriction enzyme site that encoded the junction of amino acids 708, 709 and 1477 (replacing Ile708 with Thr). In addition, this variant has an introduced MluI site at position 1544-1545 that introduces the point mutation replacing Leu1544 with a Thr. This is a consequence of the introduction of the Mlu1 site in the FV-cDNA [28].

SEQ ID No: 4
AQLRQFYVAAQGISWSYRPEPTNSSLNLSVTSFKKIVYREYEPYFKKEKP
QSTISGLLGPTLYAEVGDIIKVHFKNKADKPLSIHPQGIRYSKLSEGASY
LDHTFPAEKMDDAVAPGREYTYEWSISEDSGPTHDDPPCLTHIYYSHENL
IEDFNSGLIGPLLICKKGTLTEGGTQKTFDKQIVLLFAVFDESKSWSQSS
SLMYTVNGYVNGTMPDITVCAHDHISWHLLGMSSGPELFSIHFNGQVLEQ
NHHKVSAITLVSATSTTANMTVGPEGKWIISSLTPKHLQAGMQAYIDIKN
CPKKTRNLKKITREQRRHMKRWEYFIAAEEVIWDYAPVIPANMDKKYRSQ
HLDNFSNQIGKHYKKVMYTQYEDESFTKHTVNPNMKEDGILGPIIRAQVR
DTLKIVFKNMASRPYSIYPHGVTFSPYEDEVNSSFTSGRNNTMIRAVQPG
ETYTYKWNILEFDEPTENDAQCLTRPYYSDVDIMRDIASGLIGLLLICKS
RSLDRRGIQRAADIEQQAVFAVFDENKSWYLEDNINKFCENPDEVKRDDP
KFYESNIMSTINGYVPESITTLGFCFDDTVQWHFCSVGTQNEILTIHFTG
HSFIYGKRHEDTLTLFPMRGESVTVTMDNVGTWMLTSMNSSPRSKKLRLK
FRDVKCIPDDDEDSYEIFEPPESTVMATRKMHDRLEPEDEESDADYDYQN
RLAAALGTREFNPLVIVGLSKDGTDYIEIIPKEEVQSSEDDYAEIDYVPY
DDPYKTDVRTNINSSRDPDNIAAWNYTRSNNGNRRNYYIAAEEISWDYSE
FVQRETDIEDSDDIPEDTTYKKVVFRKYLDSTFTKRDPRGEYEEHLGILG
PIIRAEVDDVIQVRFKNLASRPYSLHAHGLSYEKSSEGKTYEDDSPEWFK
EDNAVQPNSSYTYVWHATERSGPESPGSACRAWAYYSAVNPEKDIHSGLI
GPLLICQKGILHKDSNMPMDMREFVLLFMTFDEKKSWYYEKKSRSSWRLT
SSEMKKSHEFHAINGMIYSLPGLKMYEQEWVRLHLLNIGGSQDIHVVHFH
GQTLLENGNKQHQLGVWPLLPGSFKTLEMKASKPGWWLLNTEVGENQRAG
MQTPFLIMDRDCRMPMGLSTGIISDSQIKASEFLGYWEPRLARLNNGGSY
NAWSVEKLAAEFASKPWIQVDMQKEVIITGIQTQGAKHYLKSCYTTEFYV
AYSSNQINWQIFKGNSTRNVMYFNGNSDASTIKENQFDPPIVARYIRISP
TRAYNRPTLRLELQGCEVNGCSTPLGMENGKIENKQITASSFKKSWWGDY
WEPFRARLNAQGRVNAWQAKANNNKQWLEIDLLKIKKITAIITQGCKSLS
SEMYVKSYTIHYSEQGVEWKPYRLKSSMVDKIFEGNTNTKGHVKNFFNPP
IISRFIRVIPKTWNQSIALRLELFGCDIY

As outlined above, the use of variants is also encompassed in the method of the present invention. By “FV-short variant” we mean an alternative splice variant to FV-short, which retains the functional feature of high binding affinity for TFPIα and synergistic TFPIα cofactor activity with protein S. Examples of such variants are given within the description.

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods, and can be computed even for complex mixtures by methods such as those, for example, set forth in Caceci et al. (Byte 9:340-362, 1984; the disclosures of which are incorporated herein by reference). Standard assays to evaluate the binding ability of ligands towards targets are known in the art, including for example, ELISAs, Western blots, RIAs, and flow cytometry analysis. The binding kinetics (e.g., binding affinity) of a polypeptide can also be assessed by standard assays known in the art, such as by Biacore™ system analysis.

A competitive binding assay can be conducted in which the binding of the polypeptide to a target is compared to the binding of the target by another, known ligand of that target, such as another polypeptide. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA.

Thus, the FV-short variant is preferably capable of binding to TFPIα with an affinity that is at least two-fold, 10-fold, 50-fold, 100-fold or greater than its affinity for binding to another non-target molecule. It will be appreciated that the binding to TFPIα is via the C-terminal section of the B-domain of the FV-short variant.

Accordingly, in one embodiment, by “high binding affinity” we mean the Kd of the FV-short variant is less than 1 nM.

By “functionally equivalent FV-variant” we mean a variant of FV, which has an equivalent function to FV-short, i.e. the variant has a high affinity for TFPIα (compared to non-variant FV). Binding affinity is as defined above in relation to FV-short variants. The high affinity may be due to the exposure of the acidic region in the C-terminal part of the B domain, which constitutes a high affinity-binding site for TFPIα, as described above for FV-short. In addition, the functionally equivalent FV-variants have synergistic TFPIα cofactor activity with protein S.

An example of a variant of full length FV is FV that is cleaved at Arg709 and or Arg1018 but due to a mutation of Arg1545 to Gln is not cleaved at 1545, as discussed in more detail later in the description.

By “variants” as described above we include insertions, deletions and substitutions, either conservative or non-conservative. For example, conservative substitution refers to the substitution of an amino acid within the same general class (e.g. an acidic amino acid, a basic amino acid, a non-polar amino acid, a polar amino acid or an aromatic amino acid) by another amino acid within the same class. Thus, the meaning of a conservative amino acid substitution and non-conservative amino acid substitution is well known in the art. In particular we include variants which retain the functional feature of high affinity for TFPIα.

In one embodiment, the variant has an amino acid sequence which has at least 50% identity with the amino acid sequence according to SEQ ID NO: 2, or a fragment thereof, for example at least 55%, 60%, 65%, 70%, 75%, 80%, 90%, 95%, 96%, 97%, 98% or at least 99% identity.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program [36]. The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

Alternatively, the BESTFIT program may be used to determine local sequence alignments.

The FV, FV-short, or variants as described above for use in the assay may be human or derived from other species, or artificial/synthetic.

By “FXa” (also “Factor Xa”) we mean activated coagulation Factor X (FX). The FXa utilised in the method can be human or derived from another species. Alternatively, the FXa may be artificial/synthetic. Exemplary FX includes human FX with a sequence corresponding to that of the accession number P00742 in the UniProtKB database, or bovine FX, which has the accession number P00743. These are the zymogen forms of FX and they have to be activated to the active enzyme FXa. Alternatively, the FX may be from another non-human mammal e.g. monkey, pig, mouse or rat.

The sample obtained from a subject contains unknown amounts of functional protein S.

Protein S is a vitamin K-dependent plasma protein having multiple functions. In plasma protein S is present both as free protein and in complex with C4BP. The free protein S is a cofactor to APC in the regulation of coagulation. Protein S also acts as a cofactor to tissue factor pathway inhibitor alpha (TFPIα) in inhibition of FXa. By “functional protein S levels” we include the meaning of the levels of protein S capable of acting as a cofactor to TFPIα in synergy with FV-short or FV-short variants. An exemplary protein S sequence has the UniProtKB accession number P07225.

By “functional protein S” we also include the meaning of free protein S, i.e. the protein S that is not bound in a complex. It has previously been suggested that C4BP complexed protein S also has some limited ability to act as a cofactor for TFPIα [5]. However, even if this is the case, the inventors have surprisingly found that the present assay detects only the TFPIα activity stimulated by the free, non-C4BP bound protein S fraction, as demonstrated by the Examples. Although previous assays that are reported to detect functional protein S levels via the ability of protein S to act as a cofactor for APC demonstrate an appreciable correlation between functional protein S level and healthy vs protein S deficient status [35], the same work demonstrated that when the activity of TFPIα rather than APC was assessed, the correlation was poor, and would not be suitable for medical use in, for example diagnostics, since the values obtained from protein S deficient individuals overlapped with those of healthy individuals. However, the present assay, as demonstrated herein and based on a measure of TFPIα activity, provides a measure of free or functional protein S levels that correlate well with healthy vs protein S deficient status in patients and can be used in medical diagnostics, for example.

Accordingly, in one embodiment the invention provides an in vitro method for the determination of free protein S levels in a sample. In the same or alternative embodiment the invention provides an in vitro method for the determination of non-C4BP bound protein S levels in a sample.

By “level” we include the meaning of the physical amount of protein S, for example free protein S, for example non-C4BP complexed protein S. For example, it is considered that the FXa activity typically correlates well with the physical amount of protein S, for example the amount of free protein S, for example amount of non-C4BP complexed protein S. However, in some embodiments, the term “level” is intended to mean “activity”, for example the activity of protein S in the sample, rather than the physical abundance of protein S. This is because it is expected that it is the actual amount of activity attributed to the pool of protein S (for example free protein S, for example non-C4BP complexed protein S) rather than the physical amount of free protein S (for example non-C4BP complexed protein S), that is considered to be important in determining whether a subject has sufficient protein S activity for normal function or is deficient in protein S activity. For example, and as discussed herein, in some types of protein S deficiency the actual amount of protein S protein present in the sample may be within a normal range, but the activity of protein S (for example free protein S, for example non-C4BP complexed protein S) for example as determined by the method of the invention, is low. Since in some embodiments the present invention determines the overall activity of the relevant protein S pool, the methods of the invention are suitable for diagnosing all types of protein S deficiency that result in a reduction in the TFPIα cofactor activity of protein S, for example though reduced protein abundance, and/or reduced function of protein S in terms of its TFPIα cofactor activity It is considered that in the absence of FV-Short, protein S has some intrinsic cofactor activity for TFPIα. However, the combination of protein S and FV-Short synergistically enhances the ability of protein S to act as a cofactor for TFPIα. Since the present invention detects the ability of protein S and FV-Short to synergistically enhance TFPIα and subsequently inhibit FXa activity, in some embodiments, the TFPIα cofactor activity of protein S is the cofactor activity that is synergistically enhanced in the presence of FV-short, as described herein. If necessary, to specifically detect the protein S level or activity that is synergistically enhanced by FV-Short, the skilled person understands how to perform the appropriate controls, for example by running the method of the invention in the absence of FV-short (i.e. step (a) as described herein). This will give a baseline measure of any inherent ability of protein S to act as a cofactor for TFPIα, which can then be subtracted from the ability of protein S to act as a cofactor for TFPIα in the presence of FV-Short. However, since the inherent activity of protein S is low, it is not generally considered necessary to account for this activity when trying to determine is a subject has a protein S deficiency.

However, the present invention does not detect the ability of protein S to act as a cofactor for APC and so is not suitable for detecting functional defects in APC cofactor activity.

Accordingly, in some embodiments, by activity we specifically mean the TFPIα cofactor activity of protein S (for example free protein S, for example non-C4BP complexed protein S). In these cases, the methods of the invention are suitable for use in detecting and diagnosing protein S deficiencies in a subject, wherein the deficiency at least results in a deficiency in the TFPIα cofactor activity (for example with or without a concomitant deficiency in APC cofactor activity), but is not suitable for diagnosing protein S deficiencies where the deficiency is a deficiency only in the APC cofactor activity of protein S. Although diseases that result from a protein S deficiency of only TFPIα cofactor activity have not been described, the present invention, in conjugation with an APC cofactor activity assay such as that described in [35] is capable of distinguishing between the two, should this become necessary.

The skilled person will understand then that the present method of the invention can be used to detect an overall decrease in the abundance of protein S (type I deficiencies), since a decrease in protein S will result in a decrease in TFPIα cofactor activity; and can also be used to detect a specific deficiency in TFPIα cofactor activity, regardless of protein abundance.

In some embodiments, by activity we include the meaning of the activity of the protein S to function as a cofactor for TFPIα.

Accordingly, in some embodiments, the invention provides an in vitro method for the determination of the level of protein S activity, for example free protein S activity, for example non-C4BP complexed protein S activity, for example where the activity is the TFPIα cofactor activity of protein S in a sample, wherein the method comprises the steps of:
(a) contacting a sample obtained from a subject with TFPIα and one or more of: FV-short; or an FV-short variant; or a functionally-equivalent FV-variant;
(b) contacting the sample with FXa; and
(c) measuring the level of FXa activity in the sample
wherein the level of FXa activity is indicative of the level of free (or non-C4BP complexed) protein S activity in the sample.

Accordingly, in some embodiments, the term “levels” used throughout can be taken to refer to “activity”. For example, in methods of diagnosis, the protein S activity as determined by the present invention in a test sample can be compared to a control level of protein S activity, or to a level of protein S activity obtained from one, or a number of, healthy control samples.

By “TFPIα” (also “TFPI alpha” or “TFPI”) we mean tissue factor pathway inhibitor alpha. TFPIα is an important regulator of the initial steps of the extrinsic pathway of coagulation [17, 19]. This pathway is activated in response to vascular damage and exposure of tissue factor (TF) to blood components. An exemplary TFPIα sequence is that of UniProtK accession number P10646, as given below in SEQ ID NO: 5.

>sp|P10646-2|TFPI1_HUMAN Isoform Beta of Tissue
factor pathway inhibitor OS = Homo sapiens
OX = 9606 GN = TFPI
SEQ ID NO: 5
MIYTMKKVHALWASVCLLLNLAPAPLNADSEEDEEHTIITDTELPPLKLM
HSFCAFKADDGPCKAIMKRFFFNIFTRQCEEFIYGGCEGNQNRFESLEEC
KKMCTRDNANRIIKTTLQQEKPDFCFLEEDPGICRGYITRYFYNNQTKQC
ERFKYGGCLGNMNNFETLEECKNICEDGPNGFQVDNYGTQLNAVNNSLTP
QSTKVPSLFVTKEGTNDGWKNAAHIYQVFLNAFCIHASMFFLGLDSISCL
C

In one embodiment of the first aspect of the invention, step (a) of the method further comprises contacting the sample with a substrate capable of allowing protein assembly.

By “substrate capable of allowing protein assembly” we include any substrate which enables to proteins involved in the reaction to assemble more easily than if the substrate was not present. The substrate will therefore enhance the reaction rate compared to the reaction rate of the reaction in the fluid phase.

In one embodiment, this is a substrate which allows the proteins involved in the method to assemble on the surface of the substrate, for example if the proteins in the method have affinity for the surface. Such assembly allows protein-protein interactions and increases efficiency of the reactions within the method.

The substrate capable of allowing protein assembly may be phospholipid vesicles. The proteins in the reaction have affinity for the negatively charged phospholipid membrane, and this enhances the rate of reaction.

In one embodiment step (a) also comprises calcium, or an equivalent. Equivalents include, for example, other divalent cations such as magnesium. The calcium or equivalent functions in keeping the proteins in the right conformation and active, additionally it is important for the protein-phospholipid interactions. Optionally, the calcium is present in concentrations between 0.1-30 mM, optionally between 0.1-10 mM. In one embodiment the calcium is present in concentrations between 1-2 mM.

The steps of the method may be performed sequentially, i.e. step (a), then step (b), then step (c). It will also be appreciated that the steps may be performed in another order, e.g. step (b) before step (a). It will also be appreciated that any two of, or all three of, the steps may be simultaneous. For example, in one embodiment step (a) and step (b) are performed at the same time, followed by step (c). In another embodiment, steps (a), (b) and (c) all occur at the same time.

In one embodiment, the method further comprises or consists of the steps of:
(d) providing a standard curve based on functional protein S; and
(e) comparing the measurement of step (c) to the standard curve of step (d).

As for steps (a), (b) and (c), as outlined above, it will be appreciated that the steps of the method may be performed in any order, although step (e) is dependent on steps (c) and (d) having already been performed.

In one embodiment, the standard curve of step (d) is generated using plasma samples obtained from healthy individuals. These samples can be pooled before using them as a standard. They can also be analysed individually to determine the normal range of functional protein S activity.

By “healthy individual” we include human subjects which do not have known protein S related disorders, and which have a normal level of protein S.

Alternatively, the standard curve is generated using known amounts of purified protein S, or a media solution containing a defined amount of protein S.

As a further alternative, the standard curve may be generated using protein S-deficient plasma, optionally reconstituted with known amounts of protein S.

Optionally the standard curve is generated using the same protocol as for the test sample. So the steps are performed for the same amount of time and using the same concentrations of substrates in the method.

It will be appreciated that the standard curve of step (d) can be compared to the results of step (c) to determine if the level of functional protein S in the sample is within a normal range.

Protein S is a cofactor for TFPI and enhances TFPI activity. TFPI itself is an inhibitor of FXa activity. Accordingly, it is considered that there is an inverse correlation between the level of functional protein S or the activity of protein S and the level of FXa activity within the sample. In one embodiment of the invention, the level of FXa activity measured is indicative of the inhibition of FXa, i.e. the measurement made is measuring the loss of FXa activity due to the inhibition by TFPI. The level of inhibition of FXa is indicative of the level of functional protein S in the sample, for example is indicative of the level of protein S activity in the sample, for example free protein S, for example non-C4BP complexed protein S.

By “level of FXa inhibition” we include the meaning of the decrease in activity of FXa. FXa inhibition is indicative of functional protein S because FV-short and protein S act as synergistic TFPIα cofactors, supporting the activity of TFPIα as an inhibitor of FXa. Thus, as protein S levels or activity increases, the level of FXa inhibition increases. Therefore, in this context the ‘function’ of protein S is as a synergistic TFPIα cofactor with FV-short (and FV-short variants).

It would be appreciated by one skilled in the art that FXa inhibition can be measured in a number of different ways, as disclosed herein. Level of FXa activity can be measured by, for example, a low molecular weight synthetic substrate which changes colour when FX cleaves it. The rate of substrate conversion is directly related to the actual concentration of FXa at that time. Thus, for such methods, the readout performed would be absorbance. A synthetic substrate can also use fluorescent readout.

The level of FXa can also be measured using its natural substrate prothrombin. Alternatively antibodies or other binding molecules that are specific for either inhibited or active FXa could be used to distinguish activate FXa and inhibited FXa.

In one embodiment, the method does not involve a thrombin generation assay (TGA). The skilled person will understand what is intended by the term TGA, and what assays fall under the term TGA. It is considered that FV-Short, when added to a TGA system, has a procoagulant effect, which is inseparable from any effects of protein S on TFPIα activity. Accordingly, it is considered that a TGA is not compatible with the methods of the present invention.

In one embodiment of the invention the sample is plasma. Optionally the sample is citrated plasma. By citrated plasma, we mean that calcium is chelated and therefore coagulation cannot proceed. In an alternative embodiment, plasma containing other coagulation inhibitors can be used. Examples of alternative samples include plasma containing EDTA or Li-heparin or thrombin inhibitors such as hirudin or low molecular weight synthetic thrombin inhibitors.

In one embodiment of the invention, the sample has a high dilution factor, for example wherein the dilution factor is between 1/10 and 1/2000, optionally the dilution factor is between 1/25 and 1/800, and as a further optional embodiment the dilution factor is approximately 1/50-1/400. For example, the dilution factor may be 1/50, 1/100, 1/200 or 1/400. By a dilution factor of 1/X, we mean the sample is present in a ratio of 1:X wherein X is the concentration of a dilution substrate. When the FV-short variant FV-709-1476 is used in the method, a higher dilution factor can be used, up to around 1/1000-1/2000. On the other hand, in samples with deficient protein S, a low dilution, such as a 1/10 dilution, may be required to achieve sufficient protein S effect.

One particular and unexpected advantage of the present method is that the synergistic cofactor activity between protein S and FV-short means that the assay can be operated using low concentrations of protein S. The method can detect concentrations as low as a few nM of protein S, whereas the plasma concentration of free protein S is around 100 nM.

This allows for relatively high dilution of the plasma sample used in the method, as described above. This results in the dilution of potentially disturbing or inhibitory factors from plasma, therefore minimising, or eliminating, any effect or influence of these substrates/disturbing factors on the method. To further minimise the potential coagulation activation it is possible to use thrombin inhibitors, as described herein.

In some embodiments, the dilution substrate is a buffer, thus the sample of the method is diluted in a buffer. Optionally the buffer has a pKa between 7 and 8 and is compatible with Ca²⁺.

Example buffers include HNBSACa²⁺ buffer and BSA buffer. HNBSACa2+ is a Hepes (e.g. 10-50 mM) based buffered saline (≈0.15 M NaCl) and the BSA is bovine serum albumin used as carrier protein when samples are highly diluted. An alternative buffer option is Tris-HCl buffered saline pH around 7.4 with BSA and Ca²⁺. Other example buffers which can be used in the method of the invention include MOPS, Trizma, TES, Tricine. The example buffer formulations comprise saline and Ca²⁺ and BSA or an equivalent protein carrier—for example. ovalbumin, gelatine, human albumin, PEG (poly ethylene glycol) variants or similar to minimize protein adsorption.

Accordingly, in one embodiment the method of the invention is capable of detecting low levels (or activity) of protein S, for example low levels (or activity) of functional protein S, for example low levels (or activity) of free protein S, for example low levels (or activity) of protein non-C4BP complexed protein S. In terms of physical abundance, by low levels we mean the levels of protein S in the sample may be in the range of between 0.1 and 5 nM in the diluted sample, and optionally the levels of protein S in the diluted sample are less than 3 nM.

In the undiluted sample, the levels of protein S present may be between 10 to 1000 nM, for example the protein S levels may be approximate 100 nM in the undiluted sample. In human plasma samples free protein S could be up to 200-300 nM in rare cases. The undiluted samples should be diluted to give a protein S range of between 0.1 and 5 nM as outlined above.

In one embodiment of the invention, the method further comprises contacting the sample with C4BP. Optionally this takes place in step (a) of the method.

By C4BP we mean complement regulator C4b-binding protein. In plasma C4BP circulates in complex with protein S, approximately 30% of protein S in plasma is present as free protein S, and the rest is bound to C4BP. The C4BP added to the assay/method is dissociated from protein S to expose the protein S binding site of the beta-chain of C4BP. This enables binding of the C4BP to protein S in the method.

C4BP is composed of two types of subunits, seven alpha-chains are linked by disulphide bridges to each other and to a single beta chain. The protein S binding site is on the beta-chain. The exemplary alpha chain gene has accession number P04003, whereas the exemplary beta-chain accession number is P20851.

In one embodiment of the invention step (b) further comprises contacting the sample with a component capable of emitting a measurable signal in the presence of FXa. It will be appreciated by one skilled in the art that such components can be used for the measurement of FXa activity levels, optionally for measuring FXa inhibition.

In one embodiment the measurable signal emitted in the presence of FXa is fluorescence or colour.

For example, the measurable component may be a low molecular weight synthetic substrate which changes colour when FXa cleaves it, i.e. the substrate is a chromogenic substrate. Such substrates are also referred to as “FXa substrates”. The rate of substrate conversion is directly related to the actual concentration of FXa at that time. In another embodiment, the synthetic substrate can also use fluorescent readout. These substrates may be coloured or fluorescent groups attached to small peptides. FXa cleaves the peptide and liberates the coloured or fluorescent group, and the resulting colour or fluorescence is measured. When the group is still attached it gives no colour or fluorescence.

The level of FXa can also be measured with other substrates, for example the natural substrate prothrombin.

In an alternative embodiment, antibodies or other binding molecules that are specific for either inhibited or active FXa can be used to distinguish between active FXa and inhibited FXa.

In one embodiment of the invention the measurable component is S2765, a compound with the formula: Z-D-Arg-Gly-Arg-pNA.2HCl.

Alternatively, the measurable component may be selected from the group consisting of:

| | |
|---|---|
| (i) | S-2222(Bz-Ile-Glu(γ-OR)-Gly-Arg-pNA•HCl) |
| (ii) | CH3OCO-D-CHA-Gly-Arg-pNA-AcOH |
| (iii) | Boc-Ile-Glu-Gly-Arg-AMC |
| (iv) | Boc-Leu-GLy-Arg-AMC |
| (v) | Methoxycarbonyl-D-Nle-Gly-Arg-pNA |
| (vi) | Tos-Gly-Pro-Lys-pNA |
| (vii) | Z-Lys-SBzl•HCl |
| (viii) | Mes-D-LGR-ANSN(C₂H₅)₂ |

Optionally, these components may be present as acetate salts.

In one embodiment, the concentration of the component capable of emitting a measurable signal is between 0.1 to 2 mM. Preferably the concentration is between 0.3 to 1 mM, and more preferably the concentration is 0.8 mM.

In a preferred embodiment, the component is S2765, present in the concentration ranges given above.

In one embodiment of the invention, the method further comprises contacting the sample with a thrombin inhibitor in step (a) and/or step (b). Preferably the contact with a thrombin inhibitor occurs in step (a) of the method.

By “thrombin inhibitor” we mean a substance which is capable of inhibiting the enzyme thrombin (factor IIa). Such substances act as anticoagulants through the inhibition of thrombin.

Thrombin inhibitors for use in the method disclosed herein may be bivalent, univalent or allosteric thrombin inhibitors. Bivalent inhibitors bind the active site and exosite 1 of thrombin, whereas univalent inhibitors bind only to the active site. Examples of bivalent thrombin inhibitors include: hirudin, bivalirudin, lepirudin, and desirudin. Examples of univalent thrombin inhibitors include: argatroban, inogatran, melagatran, ximelagatran, Pefabloc and dabigatran. Allosteric inhibitors include DNA aptamers, benzofuran dimers, benzofuran trimers, polymeric lignin and sulfated β-O4 lignin (SbO4L).

In one embodiment, the thrombin inhibitor is hirudin or Pefa-block (also “Pefabloc”).

In one embodiment the FV-Short variant or FV-variant used in the method is a variant which is resistant to activation by thrombin.

For example, such variants include FV-short variants and FV-variants wherein the thrombin cleavage sites are mutated from Arginine (R) to Glutamine (Q). In particular such mutants may be mutated around the positions 709 and 1545, thus affecting the cleavage sites.

One example mutant is FV-short QQ, which has both position Arg709 and position Arg1545 (numbering as in full length FV) mutated to Gln. This mutant is resistant to thrombin cleavage as the cleavage positions have both been mutated [25].

Another example mutant is FV-short RQ (also known as FV-short 1545Q) wherein Arg1545 is mutated to Gln (Q), but position 709 is unmutated and is Arg. This mutant can therefore be cleaved at Arg709 but is not cleaved at Gln1545 (as this position is resistant to thrombin cleavage). Therefore, the mutant retains the acidic region which is required for the TFPIα cofactor function (the acidic C-terminal part of the B domain). Another example mutant is FV-short QR, which can be cleaved at 1545. This mutant would therefore lose the TFPI cofactor function after cleavage. This mutant may still be used in the assay as it is not cleaved before or during the assay.

Therefore, in one embodiment the mutant FV-short is selected from FV-short QQ, FV-short RQ and FV-short QR.

Further examples of FV-short mutants include FV-709-1476 and FV-810-1491, as already described.

The sequence of the FV-short variant called FV-709-1476 is given above as SEQ ID NO: 4.

The FV-Short variant “FV-810-1491” has the deletion of amino acids 811 to 1490. This mutant binds TFPIα but has no synergistic cofactor activity with protein S [29]. This demonstrates that not all FV-Short variants can be used to construct the assay.

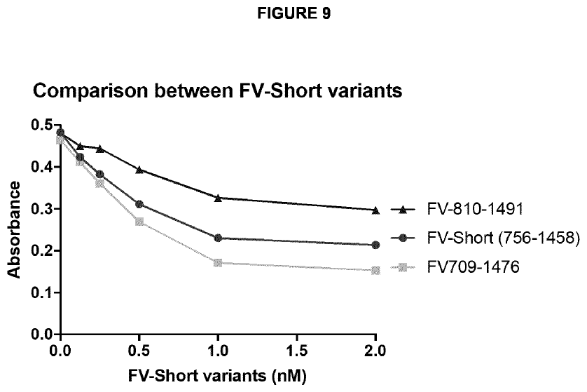

The FV-709-1476 has increased synergistic TFPIα-cofactor activity as compared to FV-Short, whereas the FV-810-1491 interestingly has little or no synergistic cofactor activity with protein S (see, e.g. FIGS. 9-11 of the examples of the present application). Thus, the sequence missing in FV 810-1491 but present in both FV-Short and in FV 709-1476 would appear to be of importance for the synergistic cofactor activities of FV-Short and FV-709-1476. This sequence corresponds to residues 1477-1490 in full length FV (EFNPLVIVGLSKDG) and is important for the synergistic TFPIα cofactor activity with protein S

Thus, in one embodiment the mutant used in the method is FV-709-1476 (SEQ ID NO: 4).

In one embodiment of the method as claimed the FV-short variant or FV variant is a variant with synergistic or enhanced cofactor activity. By “synergistic or enhanced cofactor” activity”, we mean that when protein S is not present the FV-short variant or FV variant exhibits low TFPIα cofactor activity, and therefore low levels of FXa inhibition. However, when protein S is present the FV-short variant or FV variant acts with protein S, as synergistic TFPIα cofactors, in order to inactivate FXa, leading to higher levels of FXa inhibition. Thus, protein S without FV-short or an FV-short variant present has much lower TFPIα cofactor activity and that much higher concentrations of protein S must be added to get efficient TFPIα cofactor activity.

Variants which retain synergistic cofactor activity have sufficient sequence before the acidic region to retain the activity. Such variants can be FV or FV-short variants. FV-709-1476 Is an example of a variant with synergistic cofactor activity, whereas FV-810-1491 does not have synergistic cofactor activity. Compared to the FV-810-1491 mutant, the FV709-1476 mutant has 14 amino acids more N-terminal of the 1491 site where the acid region of FV-810-1491 starts. These 14 residues are EFNPLVIVGLSKDG (position 1477-1490) as mentioned above.

Thus, in one embodiment, the FV-short variant or FV variant is a variant which retains the acidic C-terminal region of the B-domain.

In one embodiment of the method of the invention, the FV-variant is a variant which is capable of being cleaved by thrombin at positions 709 and 1018 and/or is not capable of being cleaved by thrombin at position 1545.

One example of such a mutant is referred to as FV-1545Q. This is a full length FV variant that is mutated at Arg1545 to Gln. The thrombin cleavage sites at positions 709 and 1018 are intact and sensitive to thrombin. This mutant (FV-1545Q) in the uncleaved form is similar to full length FV and in itself has no or little synergistic cofactor activity. However, after cleavage with thrombin at positions 709 and 1018 the FV exposes the acidic region, which is still attached because the Arg1545 is mutated to Gln (Q). The thrombin-cleaved FV-1545Q is an efficient cofactor [35].

In one embodiment of the invention as described, the method is specific for the free form of protein S. By “specific for the free form of protein S”, we mean the method is capable of determining the levels of free protein S, rather than levels of total protein S. By this we include the meaning of the levels of non-C4BP complexed S protein. The method does not detect any reported TFPIα activity that may be a property of the S protein when complexed with C4BP.

In one embodiment the method as described is capable of detecting protein S deficiency.

By protein S deficiency we include the meaning of below normal or healthy physical levels of protein S, or below normal levels of functional protein S activity. The skilled person will understand what is considered to be a normal or healthy level of S protein, and what is considered to be a deficient or unhealthy level of S protein.

It will therefore be appreciated that the method as described is suitable for identifying whether the subject is a patient with a protein S deficiency. By protein S deficiency we include type I protein S deficiency and type II protein S deficiency. That is, in some embodiments, the invention provides a method of diagnosing a subject as having a protein S deficiency.

Type I protein S deficiency is a deficiency wherein the protein S levels are decreased. The skilled person, for example the clinician, is aware of particular thresholds below which a subject is considered to have a protein S deficiency. The type I has particularly low free protein S (because C4BP binds as much as it can) which makes the present invention very useful. Assays for total protein S measure both free and complexed (to C4BP) protein S and have lower predictive value.

Type II protein S deficiency is characterized by a defective function of protein S. Accordingly, patients with type II protein S deficiency can have normal levels of protein S, but with low functionality of the protein S. It is considered that in these cases the protein S deficiency may be a deficiency in the TFPIα cofactor activity of protein S; the APC cofactor activity of protein S; or both the TFPIα cofactor activity of protein S and the APC cofactor activity of protein S.

Since the present invention determines the level of free protein S activity, rather than for example directly determining the amount of free protein S protein, the invention is able to detect protein S deficiency that is down to a) reduced amount of protein S (i.e. Type I protein S deficiency); and/or b) appropriate amount of protein S protein but wherein the protein S activity as TFPIα-cofactor is low or absent. Thus, the method is capable of identifying a patient with type I protein S deficiency. The method is be capable of identifying a patient with type II protein S deficiency having deficient TFPIα-cofactor activity. The method is not suitable for detecting deficiencies that present only as an APC cofactor activity deficiency.

A theorised possibility is that there may be cases of type II protein S deficiency characterised by low cofactor activity to TFPIα. Accordingly, in one embodiment the method may be capable of identifying type II protein S deficiency with defective TFPIα cofactor activity.

Type III protein S deficiency is characterized by normal levels of total protein S, but low levels of free protein S associated with reduced protein S activity. Since the present invention detects protein S activity associated only with the free pool of protein S and not the complexed protein S, the invention is also suitable for detection of Type III protein S deficiencies.

The protein S deficiency may be a heterozygous or homozygous protein S deficiency. Most patients are heterozygous and only very few cases of severely sick children have been described with homozygous deficiency.

In one embodiment, the protein S deficiency may be acquired. An acquired deficiency may be brought about by, for example, autoantibodies. Since the invention detects the level or activity of free protein S only, it is considered that the invention does not detect the level of activity of protein S bound to autoantibodies and so is appropriate for detecting protein S deficiencies that arise from the presence of anti-protein S autoantibodies. Acquired deficiencies may also be due to, for example, hepatic disease or a vitamin K deficiency. An example of a protein S deficient state caused by auto-antibodies is a condition that can occur after virus infections where the antibodies formed cross-react with protein S. One such virus infection that has been described with protein S deficiency is Varicella virus infection. AIDS has also been associated with low protein S levels. Anti-phospholipid antibody syndrome (lupus anticoagulants) has also been associated with acquired protein S deficiency.

Hepatic disease is associated with protein S deficiency because the majority of protein S is produced in the liver. Vitamin K deficiency is associated with protein S deficiency as vitamin K is required for synthesis of correct protein S. Protein S is a vitamin K-dependent protein containing the modified amino acid gamma-carboxy glutamic acid. Pregnancy is also associated with decreased levels of free protein S and it may be of interest to measure samples from pregnant women with the method of the invention.

In one embodiment the subject is a human.

In one embodiment, the subject is a patient being treated with warfarin.

In one embodiment the subject is a patient diagnosed with, or suspected of having, venous thrombo-embolic disease (VTE). Alternatively, the subject is a patient diagnosed with, or suspected of having a HIV infection, AIDS or low protein levels caused by autoantibodies against protein S. In one embodiment the patient has a vitamin K deficiency or hepatic disease. In one embodiment the subject is pregnant.

In one embodiment of the method of the invention the concentration of the FV-Short or FV-Short variant or FV variant is between 0.5 to 20 nM, optionally wherein the concentration is 2 nM.

In one embodiment of the method of the invention step (a) takes place at 37° C. for between 1 and 15 minutes, optionally wherein the time is 10 minutes. Preferably the time is less than 10 minutes.

Optionally, step (c) may take place for between 10 and 30 minutes, for example step (c) may take place for approximately 15 minutes.

In one embodiment of the invention, the ratio of TFPI alpha to FXa in the method is approximately 1:1.

In one embodiment of the method of the invention, the concentration of FXa is between 0.1 to 1 nM. In an optional embodiment the concentration of FXa is between 0.2 to 0.6 nM. Optionally the concentration of FXa is 0.3 nM.

In one embodiment of the method of the invention, the concentration of TFPI alpha is between 0.1 to 1 nM. In an optional embodiment the concentration of TFPI alpha is between 0.2 to 0.6 nM. Optionally the concentration of TFPI alpha is 0.3 nM.

It will be appreciated by those skilled in the art that the method of the invention may be performed in conjunction with a method to determine total protein S levels.

Total protein S can be measured with a variety of assays known to those skilled in the art. Such assays include assays using antibodies against protein S, such as enzyme linked immunosorbent assay (ELISA), immunoradiometric assays (IRMA), Laurell electroimmunoassay and radioimmunoassay (RIA). Another example of an assay for total protein S is Latex-based agglutination assay in automated instruments.

A second aspect of the invention is a method of treatment comprising identifying a subject with protein S deficiency using a method as described in accordance with the first aspect, and administering to said subject a therapeutic agent.

In one embodiment of the second aspect of the invention, the therapeutic agent administered to said subject is an anticoagulant therapy. Examples of anticoagulant therapies are known to the skilled person and include warfarin, and FXa inhibitors or thrombin inhibitors.

FXa inhibitors include both direct (for example, rivaroxaban, apixaban and edoxaban) and indirect (for example, fondaparinux) FXa inhibitors.

Examples of thrombin inhibitors are outlined above in the description.

According to a third aspect of the invention there is provided a kit for determining functional protein S levels in a sample. The kit of the invention may comprise any components required for performing a method according to the invention. In one embodiment, the kit comprises two of more of:
TFPIα, FXa, phospholipid vesicles, an FXa substrate (for example S2765), and an FV selected from: FV-short or an FV-short variant or a functionally equivalent FV-variant.

Optionally, the kit comprises all of TFPIα, FXa, phospholipid vesicles and an FXa substrate (for example S2765), and one of FV-short or an FV-short variant or functionally equivalent FV-variant (as described above in relation to the first aspect of the invention).

The kit may also comprise one or more components for detecting the APC cofactor activity of protein S, for example may comprise an anti-TFPIα antibody. For example, in one embodiment the kit comprises an anti-TFPIα antibody and any one or more of TFPIα, FXa, phospholipid vesicles, an FXa substrate (for example S2765), and an FV selected from: FV-short or an FV-short variant or a functionally equivalent FV-variant.

In one embodiment the kit comprises at least an anti-TFPIα antibody and a) FV selected from: FV-short or an FV-short variant or a functionally equivalent FV-variant, and/or b) FXa.

According to a fourth aspect of the invention there is provided a kit suitable for determining functional protein S levels in sample using a method according to the first aspect of the invention.

The invention also provides a therapeutic agent for use in treating a subject with a protein S deficiency, wherein the protein S deficiency has been identified using a method according to the invention, for example according to the first aspect of the invention. In some embodiments the therapeutic agent is an anticoagulant, as described herein.

The invention also provides the use of a therapeutic agent for the manufacture of a medicament for treating a protein S deficiency, wherein the protein S deficiency has been identified according to a method of the invention, for example according to a method according to the first aspect of the invention. In some embodiments the therapeutic agent is an anticoagulant, as described herein.

The invention also provides a method of diagnosing a subject as having a protein S deficiency, wherein the protein S deficiency has been identified according to a method of the invention, for example according to a method according to the first aspect of the invention.

As discussed above, the method is able to specifically detect the TFPIα cofactor activity of protein S. In view of this, it is possible in combination with performing a separate assay to detect the APC cofactor activity of protein S to determine:
a) if both cofactor activities are deficient—such a situation is expected to occur in Type I deficiencies where the physical abundance of protein S is low; Type III deficiencies where the abundance of free protein S is low; and may be expected to occur in Type II deficiencies, if both the TFPIα cofactor activity and APC cofactor activity of protein S are defective; or
b) If just one of the TFPIα cofactor activity or the APC cofactor activity of protein S are affected. Such a situation is not expected to present in Type I and Type III deficiencies.

Any of the methods of the present invention may be performed in conjunction with a method to determine the APC cofactor activity of protein S in the sample.

Accordingly, the present invention also provides a method of diagnosing a subject as having a deficiency in TFPIα cofactor activity of protein S, APC cofactor activity of protein S, or both TFPIα cofactor activity and APC cofactor activity of protein S, where the method comprises:

a) determining the level of TFPIα cofactor activity of protein S, wherein the determining is performed according to a method of the invention as described herein, for example comprising or consisting of the steps of:
(i) contacting a sample obtained from a subject with TFPIα and one or more of: FV-short; or an FV-short variant; or a functionally-equivalent FV-variant;
(ii) contacting the sample with FXa; and
(iii) measuring the level of FXa activity in the sample
wherein the level of FXa activity is indicative of the level of functional protein S in the sample; and

b) determining the level of APC cofactor activity in the sample.

By comparing the levels of both activities to control levels, it is possible to determine if just one or both of the cofactor activities are deficient. Once this information is known, suitable treatment strategies to mitigate one or both of the deficiencies can be put in place. For example, if the subject has a deficiency only in the TFPIα cofactor activity of protein S, a suitable therapeutic strategy may be put in place.

Alternatively, if only the APC cofactor activity of protein S is deficient, a suitable treatment strategy may be put in place.

It may be that the appropriate therapy for TFPIα cofactor activity deficiency and APC cofactor deficiency are the same.

If both cofactor functions are affected, then both treatment options may be appropriate.

Methods of treating a subject with the above therapies where the subject has been determined (using a method of the invention) to have a deficiency in a) only TFPIα cofactor activity of protein S; b) only APC cofactor activity of protein S; or c) both TFPIα cofactor activity of protein S and APC cofactor activity of protein S are also encompassed in the present invention.

The listing or discussion of an apparently prior-published document in this specification should not necessarily be taken as an acknowledgement that the document is part of the state of the art or is common general knowledge.

Preferred, non-limiting examples which embody certain aspects of the invention will now be described, with reference to the following figures:

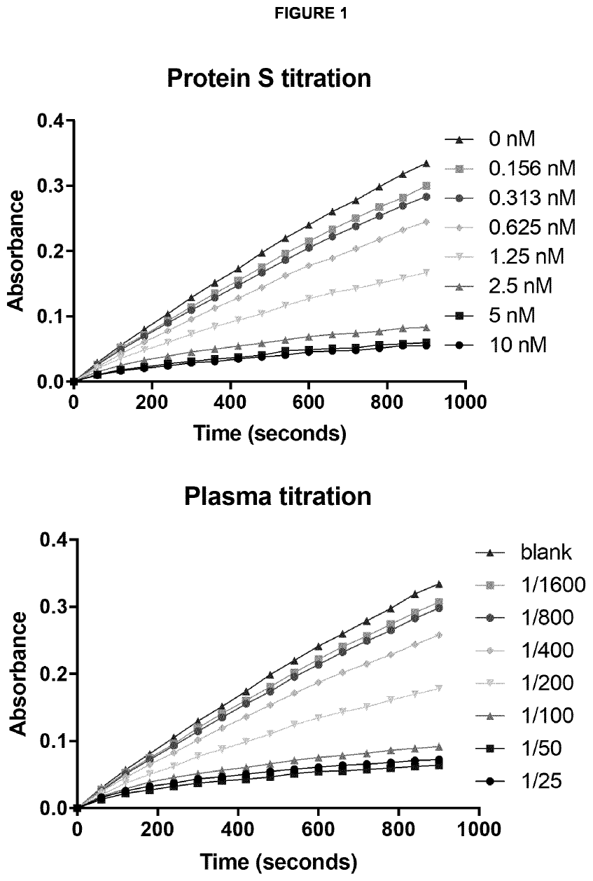

FIG. 1 Effect of protein S as cofactor to TFPIα in inhibition of FXa. FV-Short (2 nM) was incubated with FXa (0.3 nM), 25 uM PL (20/20/60:PS/PE/PC), TFPIα (0.25 nM) in the presence of different concentrations of protein S (above) or dilutions of plasma (below). The substrate (S-2765) conversion was monitored for 900 seconds and a gradually decreased slope of the curve indicates inhibition of FXa amidolytic activity.

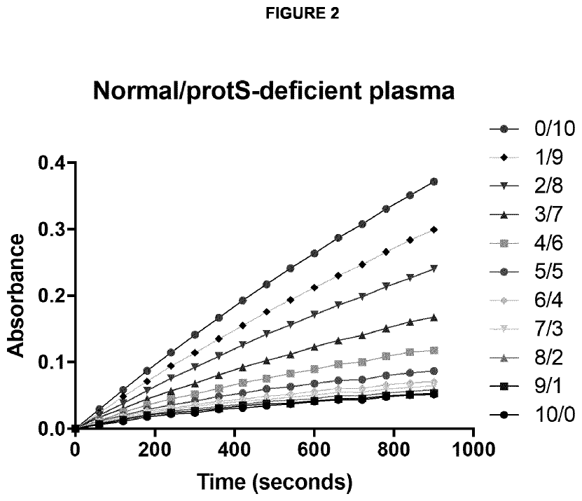

FIG. 2 Specificity for protein S as cofactor to TFPIα in inhibition of FXa. The TFPIα-mediated inhibition of FXa activity was followed as described in methods and in FIG. 1 legend. In this experiment, mixtures of normal and protein S deficient plasma was tested at a 1/100 dilution. At this dilution, normal plasma yields maximum FXa inhibition whereas protein S-deficient plasma has no activity. A dose-dependent effect is observed with increasing ratio of normal/protein S-deficient plasma.

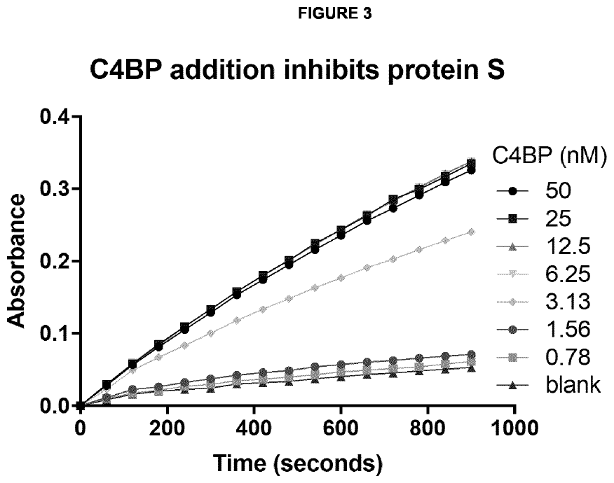

FIG. 3 Binding of C4BP to protein S results in loss of TFPIα-cofactor activity. The TFPIα-mediated inhibition of FXa (0.3 nM) by TFPIα (0.25 nM) in the presence of FV-Short (2 nM) and protein S (5 nM) was maximum resulting in a low absorbance curve. The addition of increasing concentrations of C4BP, which binds to protein S with high affinity, resulted in a dose-dependent loss of cofactor activity of protein S, at 3.13 nM C4BP much of the cofactor effect of protein S was gone and at 6.25 nM the cofactor activity was completely blocked.

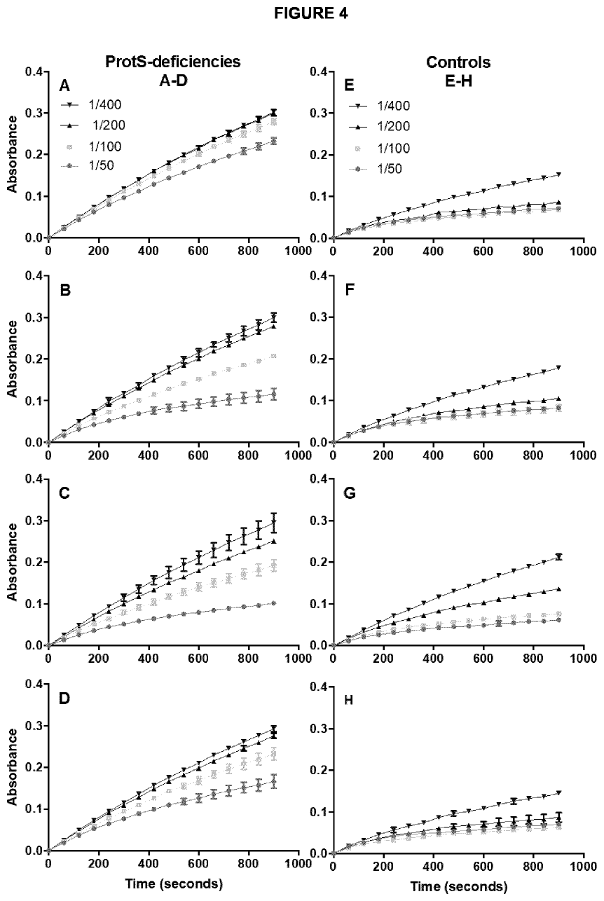

FIG. 4 Protein S deficient plasma contains little TFPIα-cofactor activity. Inhibition of FXa by TFPIα (0.25 nM) in the presence of FV-Short (2 nM) and dilutions of plasma from either controls (E, F, G and H) or protein S-deficient individuals (A, B, C and D) was monitored with synthetic substrate S-2765

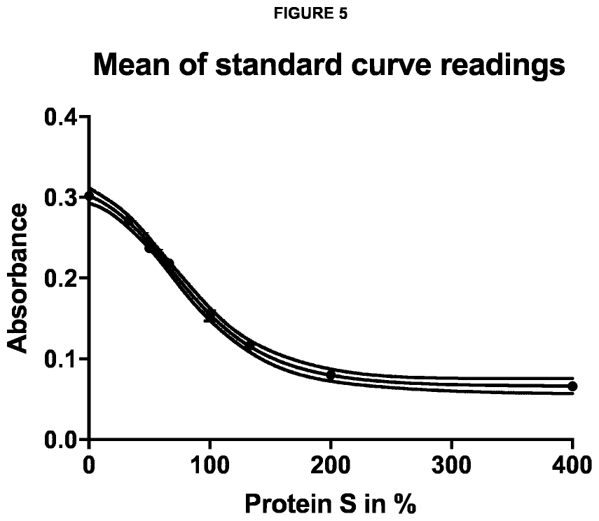

FIG. 5 Example of standard curve for TFPIα cofactor activity of protein S in plasma. The FXa inhibition by TFPIα plus FV-Short in the presence of different dilutions of normal plasma as source of protein S was monitored for 900 seconds. The final absorbance was used to construct a standard curve where 100% represents the 1/200 dilution, 50% the 1/400 dilution etc.

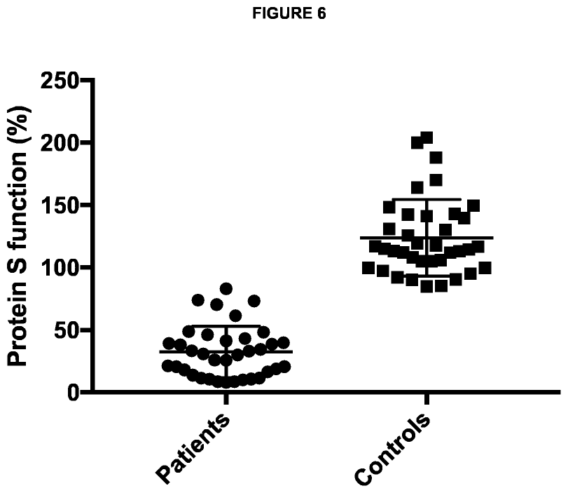

FIG. 6 Functional protein S levels in plasma of protein S deficient patients and controls. The results of the assay for TFPIα cofactor activity of plasma protein S from controls and individuals with protein S deficiency demonstrated good separation in protein S values between those with protein S deficiency (left) and those without protein S deficiency (right).

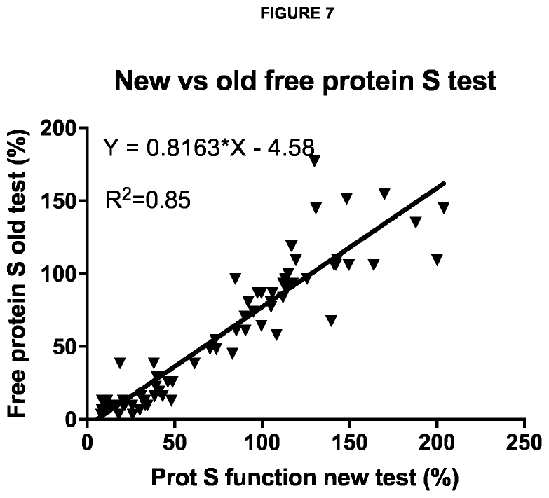

FIG. 7 Correlation between old test for free protein S and the new functional assay. The results of the new test were compared with those previously determined in an immunological free protein S assay.

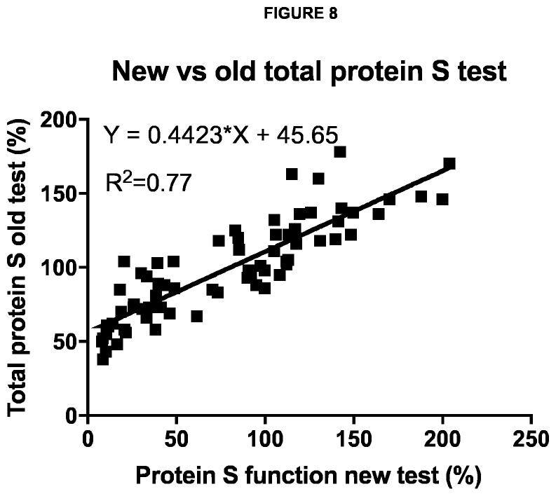

FIG. 8 Correlation between old test for total protein S and the new functional assay. The results of the new test were compared with those previously determined in an immunological total protein S assay.

FIG. 9 shows a comparison of FV short and FV-short variants. The concentration of the FV-variants are varied whereas the protein S (3 nM), TFPIα (0.25 nM) and FXa (0.3 nM) are constant. The values plotted here are the absorbances reached after 900 seconds.

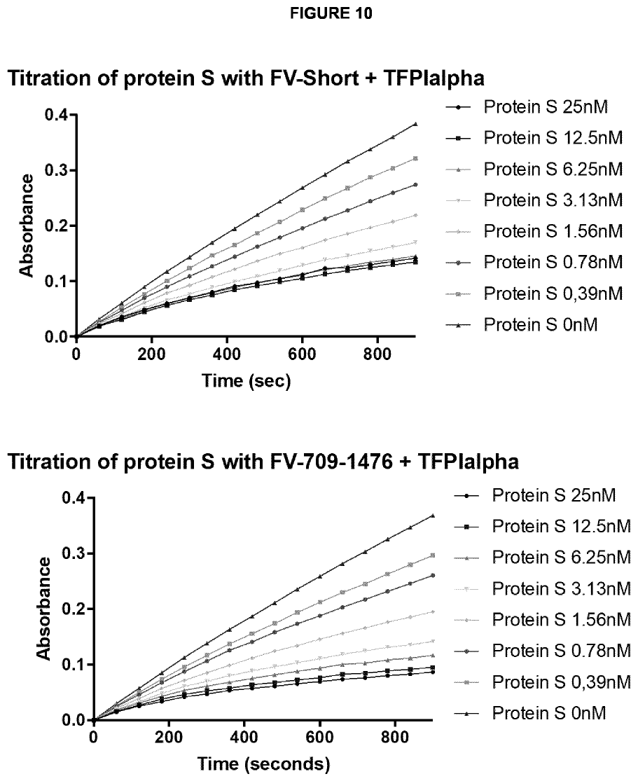

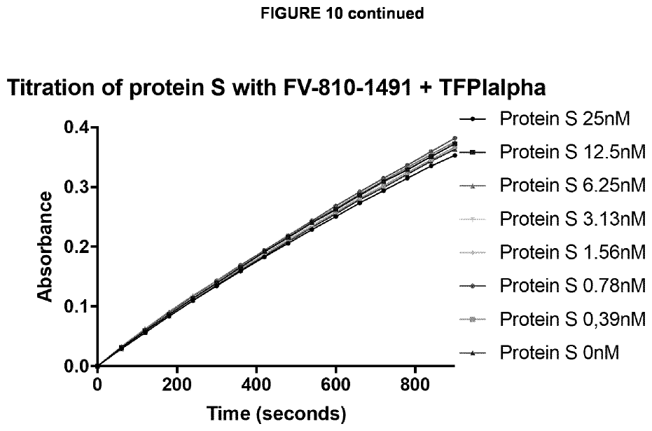

FIG. 10 shows the time curves of substrate development with the three mutants. the concentration of the FV-variants is 1 nM, the FXa (0.3 nM) and TFPIα (0.25 nM) are constant as is the FV-variant concentration. The protein S concentration is varied.

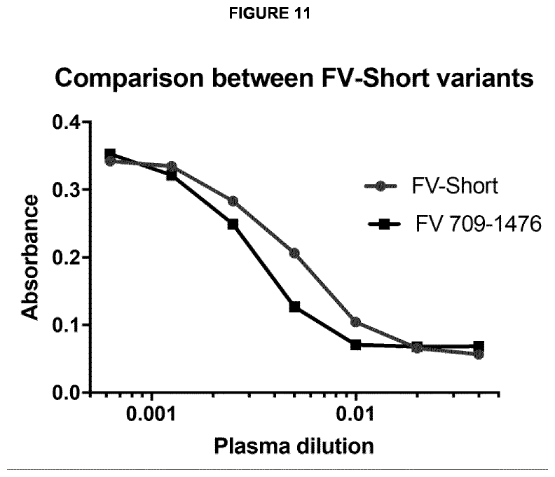

FIG. 11 shows a comparison of FV-short and FV 709-1476, using plasma dilutions as the source of protein S.

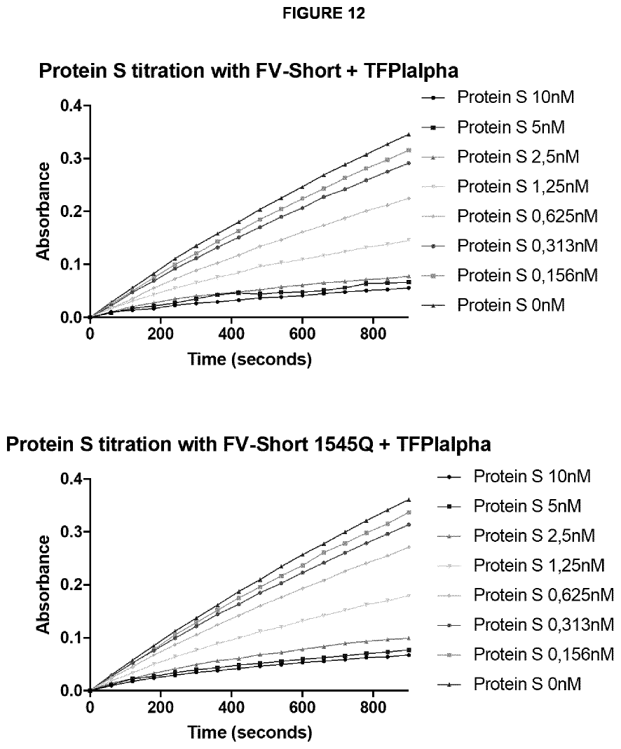

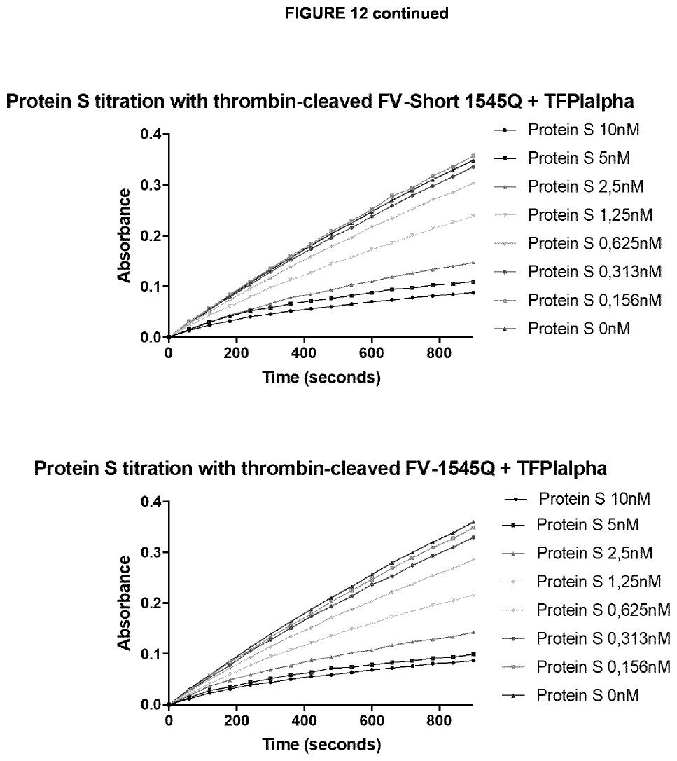

FIG. 12 shows protein S titrations of FV-Short, FV-Short 1545Q, thrombin cleaved FV-Short 1545Q and thrombin cleaved FV-1545Q, all with TFPIα.

### EXAMPLES

### Example 1—Assay for Functional Protein S

### Summary

The TFPIα-mediated inhibition of FXa in the presence of FV-Short, protein S and negatively charged phospholipid vesicles was monitored in time by synthetic substrate S2765. Diluted plasma was used as source for protein S and a standard curve was constructed using plasma dilutions

Materials and Methods

Patients—Individual citrated plasma samples (n=36) from different protein S-deficient families previously characterized were available in the laboratory[26]. Four individuals treated with warfarin with other inherited anticoagulant protein deficiencies were also available; three protein C deficiencies and one antithrombin deficiency. The samples had been stored at −80° C. since the time of collection during the 90-ties. Samples from selected from healthy family (n=37) members with no protein S deficiency or history of thrombosis were used as controls. The values for the plasma concentrations of total and free protein S were available from the published study[26]. The method to determine the free and total protein S in the samples was described previously[27]. A citrated plasma pool collected from healthy individuals was used to create the standard curve, setting the functional protein S concentration in the pool to be 100%.

Materials—Human FXa was from Hematologic Technologies, Inc (HTI); Protein S-deficient plasma was from Emzyme Research Laboratories; TFPIα expressed in eukaryotic cells was a gift from Dr T Hamuro at the Chemo-Sero-Therapeutic Research Institute, Japan. FV-Short was expressed and purified as previously described[25]-FV709-1476[28] and FV-810[29] was expressed and purified with similar technique. Phosphatidylserine (PS), phosphatidyl ethanolamine (PE) and phosphatidyl choline (PC) were from Avanti Polar Lipids. Phospholipid vesicles were prepared using the LiposoFast basic extruder (Armatis, Germany) as previously described[30]. The phospholipid vesicles were used within 2 days. Synthetic substrate S2765 was provided by Chromogenix Ltd, Milan, Italy. C4BP without bound protein S was purified as described[31].

Assay for protein S-mediated TFPIα-cofactor activity—The assay is based on the inhibition of FXa by TFPIα in a purified system using a technique we previously described[25]. In this assay, FV-Short (2 nM final concentration) was incubated for 10 minutes at 37° C. with phospholipid (20:20:60 of PS:PE:PC, 25 uM), TFPIα (0.25 nM final concentration), plasma diluted in HNBSACa²⁺ buffer (25 Hepes, 0.15 M NaCl, 5 mM CaCl₂) pH 7.7, containing 0.5 mg/ml bovine serum albumin and 5 Units/ml of Hirudin) as source of protein S. The reaction was initiated by addition of S2765 (0.8 mM) and FXa (0.3 nM) and followed for 15 minutes by monitoring absorbance at 405 nM in a Tecan Infinite 200 system. The concentrations given in each experiment are the final concentrations.

Inhibition of protein S function as TFPIα cofactor by C4BP. To investigate whether both free and C4BP-bound protein S functions as TFPIα cofactor, increasing concentrations of purified C4BP (0-50 nM) were added in a FXa inhibition assay containing 5 nM protein S.

Results

The inhibition of FXa by TFPIα in the presence of negatively charged phospholipid vesicles, FV-Short and either purified protein S or diluted pooled plasma was followed in time (FIG. 1). In the absence of protein S or plasma the absorbance curve was almost linear suggesting that there was very little inhibition of FXa by TFPIα in the presence of FV-Short alone. Addition of increasing concentrations of protein S gave a dose-dependent inhibition of FXa with 50% inhibition at 1.25 nM and maximal inhibition at 5 nM protein S. Similarly, the inclusion of diluted plasma in the assay instead of protein S yielded a dose-dependent inhibition with maximum inhibition observed at 1/50 dilution and around 50% inhibition at 1/200. The TFPIα-cofactor effect of protein S in the assay depended on the presence of FV-Short and in the absence of added FV-Short, neither protein S (up to 10 nM) nor plasma (up to 1/100 dilution) yielded any stimulation of FXa inhibition.

The assay was specific for protein S because addition of protein S-deficient plasma gave no stimulation of FXa inhibition (FIG. 2). Mixtures of normal and protein S deficient plasma were included in the assay at a 1/100 dilution. Increasing the ratio of normal/protein S-deficient plasma resulted in a dose-dependent increased FXa inhibition and close to 50% inhibition was observed at the 3:7 ratio, which provides around the same amount of protein S to the assay as the 1/200 dilution of normal plasma seen in FIG. 1.

To investigate whether both free and C4BP-complexed forms of protein were active as TFPIα cofactors, increasing concentrations of C4BP (0-50 nM) were included in reactions containing 5 nM protein S (FIG. 3). In the absence of added C4BP, the protein S-mediated stimulation of FXa inhibition was maximal. Addition of increasing concentrations of C4BP yielded a dose-dependent increased absorbance suggesting blockage of the protein S TFPIα-cofactor activity and at 6.25 nM C4BP, no protein S effect was observed. This suggests that the formation of the 1:1 stoichiometric complex of protein S and C4BP results in loss of the TFPIα-cofactor activity.

A cohort of 36 patients with known inherited protein S deficiency and 37 age and sex matched healthy controls identified from previous family studies was tested in the assay. FIG. 4 illustrates the absorbance readings from four protein S-deficient individuals and four healthy controls. The plasma to be tested was diluted 1/50, 1/100, 1/200 and 1/400 to cover the range between normal concentrations of protein S to the low protein S levels in protein S deficiency.

The final readings at 900 seconds from an assay using diluted plasma as source of protein S was utilized to construct a standard curve for quantifying the protein S activity as a TFPIα cofactor (FIG. 5). As the 1/200 dilution yielded around 50% inhibition it was set to be 100%. Consequently, the 1/400 reading corresponded to 50% and the 1/100 dilution to 200%. The best absorbance reading range was between 0.1 and 0.25 and as both patients and controls were analyzed at several dilutions, it was possible to get readings within that range.

The results from the testing of the protein S-deficient individuals and the controls are illustrated in FIG. 6. There was a separation in functional protein S values between the patients and the controls. The mean±SD values of patients and controls were 35±20 and 120±25, respectively. The values for the patients ranged between 8 and 83 and those of controls between 85 and 186. The correlation between the two assays was high with an r-value of 0.93, the slope being 0.82 and the Y-intercept −4.6. This suggests that the assay is measuring the activity of the free form of protein S.

The functional protein S values were also correlated to the total protein S values (FIG. 8). The correlation (r-value of 0.88) was lower than that to free protein S. The slope was 0.44 and the Y-intercept 46. These results are in agreement with the conclusion that the synergistic TFPIα-cofactor activity is solely associated with the free form of protein S.

Four of the protein S deficient cases were treated with warfarin. The mean±SD of their functional protein S values was 10.9±4%; range 8.0-16.6%, which agrees well with the results of the free protein S assay (8.9±4%; range 3.2-12.9). Four patients with other inherited anticoagulant deficiencies (three protein C deficiencies and one antihrombin deficiency) were tested to elucidate the effect of warfarin treatment on cases with no protein S abnormality. The mean±SD functional protein S value of these cases was 63.0±23%; range 34.3-98.9, whereas the mean±SD free protein S value was 39.5±19.6%; range 22.6-67.7%. This suggests that the TFPIα-functional test is equally efficient to detect protein S deficiency also in warfarin-treated cases, as is the free protein S assay[26].

To estimate the intra assay variation of the new test, one normal and one protein S deficient case was analyzed nine times. The mean±SD of the normal case was 85.4±4.3%; range 77.2-92.4%. Corresponding values for the protein S deficient case was 47.8±5.4%; range 40.0-53.7%. Thus, the intra assay coefficient of variation for samples with normal protein S levels was 5.1%, whereas for samples with protein S deficiency it was 10.6%.

### Discussion

Vitamin K-dependent protein S is a multi-functional plasma protein[2]. It is important as anticoagulant regulator of several reactions of blood coagulation. As cofactor to APC, it controls the activity of the cofactors in the tenase (FVIIIa) and the prothrombinse (FVa) complexes. In addition, it serves as a cofactor to TFPIα in the regulation of free FXa[2, 15, 32]. The recent observation that the TFPIα cofactor activity of protein S is stimulated by FV-Short and that FV-Short and protein S function in synergy has added to the complexity but does also provide an opportunity to devise an assay for the function of plasma protein S as TFPIα cofactor[3, 25]. We now report on the creation and characterization of such a functional protein S assay that is based on the rate of inhibition of FXa by TFPIα in the presence of FV-Short, protein S from plasma samples and negatively charged phospholipid vesicles.

The synergistic TFPIα-cofactor activity of protein S was strictly confined to the free form of protein S. This is consistent with the binding site for C4BP on protein S being located in the SHBG-like region of protein S, a region also known to interact with TFPIα[1, 2, 16, 33]. The APC-cofactor activity of protein S is also preferentially expressed by the free form of protein S and several regions in protein S, including the Gla-domain, the TSR, EGF-domains and the SHBG-like region, have been shown to be important for the APC-cofactor activity. As the TFPIα-mediated FXa-inhibitory reaction takes place on negatively charged phospholipids, the Gla domain of protein S is expected to be important for the TFPIα-cofactor activity of protein S. This is consistent with the low functional protein S levels of several non-protein S deficient warfarin-treated patients.

The C4BP binds protein S with high affinity, which explains why a decrease in plasma levels of protein S, e.g. in inherited protein S deficiency, preferentially is reflected in decreased levels of free protein S. Therefore, assays for free protein S are superior to those for total protein S for the diagnosis of protein S deficiency[26]. However, the assays for free protein S are not able to detect functional protein S deficiency. Assays for the APC-cofactor activity of protein S have been shown to detect cases with functional defects in the ACP-cofactor activity of protein S, so called type II protein S deficiencies. The now described assay for the function of protein S as TFPIα cofactor will be suitable for detecting not only the level of free protein S but also its functional activity as TFPIα cofactor and type II cases with defects in the TFPIα-cofactor function. Recently, another functional assay for protein S as TFPIα cofactor was described[34]. The assay is based on a TF-initiated thrombin-generation assay in which a fixed amount of TFPIα is added to mixtures of protein S-deficient and patient plasma. This assay is conceptually different from the one we now describe because it does not take advantage of the synergistic TFPIα-cofactor activity between protein S and FV-Short as the amount of any intrinsic FV-Short is too low in the assay. The authors found that the assay detected most cases of protein S deficiency but that the correlation to total and free protein S was relatively low.

The assay now described is specific for protein S as demonstrated by the lack of TFPIα-cofactor activity of protein S deficient plasma. The assay requires low concentrations (<3 nM) of protein S and a 1/100 dilution of normal plasma yields maximum TFPIα-cofactor activity in the presence of 2 nM FV-Short. Without the addition of the FV-Short, there is little or no TFPIα-cofactor activity at such dilutions of plasma. To detect very low levels of protein S in plasma from protein S deficient patients, lower dilution factor (1/25 or 1/50) can be used. Plasmas from previously characterized protein S deficient families were tested in the new assay and the results compared with the concentrations of both free and total protein S determined with immunological tests. The results of the new functional assay correlated well with the free protein S concentrations an r-value of 0.92. The correlation line had a Y-axis intercept of close to 0 and the slope of the line was slightly below 1. The correlation to total protein S was slightly lower with an r-value of 0.88. Interestingly, the Y-axis intercept was around 46% and the slope of the line was 0.44. The high intercept is consistent with the results showing that the TFPIα-cofactor assay is specific for free protein S.

The new functional test accurately detected the protein S-deficient cases with a good separation between those with and those without protein S deficiency. In four cases, the values were very close to the lower normal values. These four cases were from families where the protein S-deficient cases had relatively high free protein S levels. One such family is called family 18 in a previous publication and two of the borderline cases were from this family[26]. The other two borderline cases were from two other families with similar phenotype.

In conclusion, we now describe a new test for the TFPIα-cofactor activity of plasma protein S, which utilizes the recently described synergy between protein S and FV-Short. The test is specific for the TFPIα-cofactor activity of free protein S and discriminates between cases with inherited protein S deficiency and those with normal protein S level and activity. In addition, the test should be able to detect type II protein S deficiency having defective TFPIα-cofactor activity. Such patients are yet to be identified.

### Example 2—Properties of FV-Variants and FV-Short Variants

There are several FV-Short variants that we have been characterized in terms of functional activity. Two such mutants are called FV-709-1476 and FV-810-1491 (as described herein). The FV-709-1476 has increased synergistic TFPIα-cofactor activity as compared to FV-Short (also referred to as FV-756-1458), whereas the FV-810-1491 interestingly has no synergistic cofactor activity with protein S.

This is illustrated in FIG. 9, where the concentration of the FV-variants are varied whereas the protein S (3 nM), TFPIα (0.25 nM) and FXa (0.3 nM) concentrations are constant. The values plotted here are the absorbances reached after 900 seconds. FV-709-1476 is slightly more efficient than FV-Short whereas the FV-810-1491 is less efficient.

FIG. 10 shows the time curves of substrate development with the three mutants. in this experiment, the concentration of the FV-variants is 1 nM. The FXa (0.3 nM) and TFPIα (0.25 nM) concentrations are constant as is the FV-variant concentration. The protein S concentration is varied. FIG. 10 shows that the FV-709-1476 is somewhat more efficient as the protein S added has stronger stimulating effect. This has been a consistent finding throughout the experiments. Of particular note is the lack of effect of protein S with the FV-810-1491 showing that this mutant has no synergistic effect with protein S.

FIG. 11 shows a similar comparison, made using plasma dilutions as source of protein S. FIG. 11 shows the absorbances from the 900 sec point. FIG. 11 shows that the plasma can be diluted approximately twice as much with the FV-709-1476 variant as compared to the FV-Short variant, thus illustrating that the FV-709-1476 variant has higher activity.

FIG. 12 shows the activity of variant FV-Short 1545Q (resistant to thrombin cleavage at position 1545 because the Arg (R) is replaced with a Gln (Q). In the illustrated time course of FIG. 12, it is evident that this variant is essentially similar to FV-Short and moreover that the activity remains after incubation of the mutant with thrombin. The FV-Short 1545Q mutant will be cleaved at Arg709 but the acidic C-terminal part of the B domain is still attached and thus the mutant retains the synergistic cofactor activity.

The other variant shown in FIG. 12 is a full length FV variant (bottom part of the figure, on the second page of FIG. 12) that is also mutated at Arg1545 to Gln, referred to as FV-1545Q [35]. The thrombin cleavage sites at positions 709 and 1018 are intact and sensitive to thrombin. This mutant (FV-1545Q) in the uncleaved form is similar to full length FV and in itself has no or little synergistic cofactor activity [25]. However, after cleavage with thrombin (at 709 and 1018) the FV exposes the acidic region that is still attached because the Arg1545 is mutated to Gln (Q). From the time curves it is evident that that the thrombin-cleaved FV-1545Q is equally as efficient as the other variants.

### REFERENCES

1. Dahlback, B. (2007) The tale of protein S and C4b-binding protein, a story of affection. Thromb Haemost 98 (1), 90-6.
2. Dahlback, B. (2018) Vitamin K-Dependent Protein S: Beyond the Protein C Pathway. Semin Thromb Hemost 44 (2), 176-184.
3. Dahlback, B. (2017) Novel insights into the regulation of coagulation by factor V isoforms, tissue factor pathway inhibitoralpha, and protein S. J Thromb Haemost 15 (7), 1241-1250.
4. Hackeng, T. M. et al. (2009) Regulation of TFPI function by protein S. J Thromb Haemost 7 Suppl 1, 165-8.
5. Hackeng, T. M. et al. (2006) Protein S stimulates inhibition of the tissue factor pathway by tissue factor pathway inhibitor. Proc Natl Acad Sci USA 103 (9), 3106-11.
6. Dahlback, B. (2008) Advances in understanding pathogenic mechanisms of thrombophilic disorders. Blood 112 (1), 19-27.
7. Sailer, F. et al. (2009) Generation and phenotypic analysis of protein S-deficient mice. Blood 114 (11), 2307-14.
8. Burstyn-Cohen, T. et al. (2009) Lack of protein S in mice causes embryonic lethal coagulopathy and vascular dysgenesis. J Clin Invest 119 (10), 2942-53.
9. Dahlback, B. (2018) Treating hemophilia by targeting protein S? Blood 131 (12), 1271-1272.
10. Prince, R. et al. (2018) Targeting anticoagulant protein S to improve hemostasis in hemophilia. Blood 131 (12), 1360-1371.
11. Dahlback, B. (2000) Blood coagulation. Lancet 355 (9215), 1627-32.
12. Dahlback, B. and Villoutreix, B. O. (2005) Regulation of blood coagulation by the protein C anticoagulant pathway: novel insights into structure-function relationships and molecular recognition. Arterioscler Thromb Vasc Biol 25 (7), 1311-20.
13. Ndonwi, M. and Broze, G., Jr. (2008) Protein S enhances the tissue factor pathway inhibitor inhibition of factor Xa but not its inhibition of factor VIIa-tissue factor. J Thromb Haemost 6 (6), 1044-6.
14. Ndonwi, M. et al. (2010) The Kunitz-3 domain of TFPI-alpha is required for protein S-dependent enhancement of factor Xa inhibition. Blood 116 (8), 1344-51.
15. Peraramelli, S. et al. (2012) TFPI-dependent activities of protein S. Thromb Res 129 Suppl 2, S23-6.
16. Reglinska-Matveyev, N. et al. (2014) TFPI cofactor function of protein S: essential role of the protein S SHBG-like domain. Blood 123 (25), 3979-87.
17. Broze, G. J., Jr. and Girard, T. J. (2012) Tissue factor pathway inhibitor: structure-function. Front Biosci (Landmark Ed) 17, 262-80.
18. Maroney, S. A. and Mast, A. E. (2015) New insights into the biology of tissue factor pathway inhibitor. J Thromb Haemost 13 Suppl 1, S200-7.
19. Mast, A. E. (2016) Tissue Factor Pathway Inhibitor: Multiple Anticoagulant Activities fora Single Protein. Arterioscler Thromb Vasc Biol 36 (1), 9-14.
20. Vincent, L. M. et al. (2013) Coagulation factor V(A2440G) causes east Texas bleeding disorder via TFPIalpha. J Clin Invest 123 (9), 3777-87.
21. Camire, R. M. (2016) Rethinking events in the haemostatic process: role of factor V and TFPI. Haemophilia 22 Suppl 5, 3-8.
22. Ndonwi, M. et al. (2012) The C-terminus of tissue factor pathway inhibitor alpha is required for its interaction with factors V and Va. J Thromb Haemost 10 (9), 1944-6.
23. Wood, J. P. et al. (2013) Tissue factor pathway inhibitor-alpha inhibits prothrombinase during the initiation of blood coagulation. Proc Natl Acad Sci USA 110 (44), 17838-43.
24. Wood, J. P. et al. (2014) Biology of tissue factor pathway inhibitor. Blood 123 (19), 2934-43.
25. Dahlback, B. et al. (2017) Factor V-short and protein S as synergistic tissue factor pathway inhibitor (TFPIa) cofactors. Res Pract Throm Haemost 2, 114-124.
26. Zoller, B. et al. (1995) Evaluation of the relationship between protein S and C4b-binding protein isoforms in hereditary protein S deficiency demonstrating type I and type III deficiencies to be phenotypic variants of the same genetic disease. Blood 85 (12), 3524-31.
27. Malm, J. et al. (1988) Changes in the plasma levels of vitamin K-dependent proteins C and S and of C4b-binding protein during pregnancy and oral contraception. Br J Haematol 68 (4), 437-43.
28. Marquette, K. A. et al. (1995) The factor V B-domain provides two functions to facilitate thrombin cleavage and release of the light chain. Blood 86 (8), 3026-34.
29. Toso, R. and Camire, R. M. (2004) Removal of B-domain sequences from factor V rather than specific proteolysis underlies the mechanism by which cofactor function is realized. J Biol Chem 279 (20), 21643-50.
30. Happonen, K. E. et al. (2016) The Gas6-Axl Protein Interaction Mediates Endothelial Uptake of Platelet Microparticles. J Biol Chem 291 (20), 10586-601.
31. Dahlback, B. et al. (1983) Visualization of human C4b-binding protein and its complexes with vitamin K-dependent protein S and complement protein C4b. Proc Natl Acad Sci USA 80 (11), 3461-5.
32. Hackeng, T. M. and Rosing, J. (2009) Protein S as cofactor for TFPI. Arterioscler Thromb Vasc Biol 29 (12), 2015-20.
33. Somajo, S. et al. (2015) Amino acid residues in the laminin G domains of protein S involved in tissue factor pathway inhibitor interaction. Thromb Haemost 113 (5), 976-87.
34. Alshaikh, N. A. et al. (2017) New functional assays to selectively quantify the activated protein C- and tissue factor pathway inhibitor-cofactor activities of protein S in plasma. J Thromb Haemost 15 (5), 950-960.
35. Thorelli E, Kaufman R J and Dahlbäck B (1997) Cleavage requirement for activation of FV by factor Xa, Eur J Biochem. 247:12-20.
36. Thompson et al. (1994) CLUSTAL W: improving the sensitivity of progressive multiple sequence alignment through sequence weighting, position-specific gap penalties and weight matrix choice. Nuc. Acid Res. 22:4673-4680
37. Caceci. M. S. & Cacheris. W. P. (1984) Fitting curves to data. Byte 9:340-362

1. An in vitro method for the determination of functional protein S levels in a sample, the method comprising or consisting of the steps of:
(a) contacting a sample obtained from a subject with TFPIα and one or more of: FV-short; or an FV-short variant; or a functionally-equivalent FV-variant;
(b) contacting the sample with FXa; and
(c) measuring the level of FXa activity in the sample
wherein the level of FXa activity is indicative of the level of functional protein S in the sample.

2. The method according to claim 1, wherein the functional protein S level is the level of activity of protein S in the sample, optionally wherein the activity is the ability of the protein S to function as a cofactor for TFPIα.

3. The method according to claim 1, wherein the functional protein S level is the amount of functional protein S in the sample.

4. The method according to claim 1, wherein the functional protein S in the sample is the free protein S in the sample.

5. The method according to claim 1, wherein the functional protein S in the sample is the non-C4BP complexed protein S in the sample.

6. The method according to claim 1, wherein step (a) further comprises contacting the sample with a substrate capable of allowing protein assembly.

7. The method according to claim 6, wherein the substrate capable of allowing protein assembly is phospholipid vesicles.

8. The method according to claim 1, wherein step (a) further comprises calcium.

9. The method according to claim 1, further comprising or consisting of the steps of;
(d) providing a standard curve based on functional protein S; and
(e) comparing the measurement of step (c) to the standard curve of step (d).

10. The method according to claim 9, wherein the standard curve is generated using plasma samples obtained from healthy individuals, or using known amounts purified protein S, or using a media solution containing a defined amount of protein S.

11. The method according to claim 1, wherein the level of FXa activity measured is indicative of the inhibition of FXa, and wherein the level of inhibition of FXa is indicative of the level of functional protein S in the sample, optionally the level of functional protein S activity in the sample.

12. The method according to claim 1, wherein the sample is plasma, optionally wherein the sample is citrated plasma.

13. The method according to claim 1, wherein the sample has a high dilution factor, for example wherein the dilution factor is between 1/10 and 1/2000, optionally wherein the dilution factor is between 1/50 and 1/400.

14. The method according to claim 1, wherein step (a) further comprises contacting the sample with C4BP.

15. The method according to claim 1, wherein step (b) further comprises contacting the sample with a component capable of emitting a measurable signal in the presence of FXa.

16. The method according to claim 11, wherein the measurable signal emitted in the presence of FXa is fluorescence or colour, optionally wherein the component selected from: S2765, S-2222.

17. The method according to claim 16, wherein the component capable of emitting a measurable signal is S2765, and the concentration of S2765 is between 0.1 to 2 mM, preferably between 0.3 to 1 mM, preferably wherein the concentration of S2765 is 0.8 mM.

18. The method according to claim 1, wherein step (a) and/or step (b) further comprises contacting the sample with a thrombin inhibitor.

19. The method according to claim 18, wherein the thrombin inhibitor is hirudin or Pefa-block.

20. The method according to claim 1, wherein the FV-Short variant or FV-variant is a variant which is resistant to activation by thrombin.

21. The method according to claim 20, wherein the FV short variant or FV-variant contains thrombin cleavage sites which are mutated from Arginine to Glutamine, optionally wherein the FV-short variant is selected from FV-short QQ, FV-short RQ and FV-short QR.

22. The method according to claim 1, wherein the FV-short variant or FV variant is a variant with enhanced or synergistic TFPIα cofactor activity.

23. The method according to claim 22, wherein the FV-short variant is FV-709-1476.

24. The method according to claim 1, wherein the FV-short variant or FV variant is a variant which retains the acidic C-terminal region of the B-domain.

25. The method according to claim 1, wherein the FV-variant or FV-short variant is capable of being cleaved by thrombin at positions 709 and 1018 and/or is not capable of being cleaved by thrombin at position 1545.

26. The method according to claim 25, wherein the FV-variant is FV-1545Q.

27. The method according to claim 1, wherein the method is specific for the free form of protein S.

28. The method according to claim 1, wherein the method is capable of detecting protein S deficiency.

29. The method according to claim 1, for identifying whether the subject is a patient with a protein S deficiency

30. A method of diagnosing a subject as having a protein S deficiency wherein the method comprises determining the level of protein S, optionally level of protein S activity, according to claim 1.

31. The method according to claim 30, wherein the protein S deficiency is a type I protein S deficiency.

32. The method according to claim 30, wherein the protein S deficiency is a type II protein S deficiency.

33. The method according to claim 32, wherein the protein S deficiency is a type II protein S deficiency with defective TFPIα cofactor activity.

34. The method according to claim 30, wherein the protein S deficiency is a type III protein S deficiency.

35. The method according to claim 30, wherein the protein S deficiency is a heterozygous or homozygous protein S deficiency.

36. The method according to claim 30, wherein the protein S deficiency is acquired.

37. The method according to claim 30, wherein the subject is a patient being treated with warfarin.

38. The method according to claim 30, wherein the subject is a patient diagnosed with, or suspected of having, venous thrombo-embolic disease (VTE).

39. The method according to claim 1, wherein the method is capable of detecting low levels of protein S for example wherein the levels of protein S are between 0.1 and 5 nM in a diluted sample, optionally wherein the protein S levels are <3 nM in a diluted sample.

40. The method according to claim 1, wherein the concentration of the FV-Short or FV-Short variant or FV variant is between 0.5 to 20 nM, optionally wherein the concentration is 2 nM.

41. The method according to claim 1, wherein step (a) takes place at 37° C. for between 1 and 15 minutes, optionally wherein the time is 10 minutes.

42. The method according to claim 1, wherein step (c) takes place for between 10 and 30 minutes, optionally wherein the time is 15 minutes.

43. The method according to claim 1, wherein the sample is diluted in a buffer, optionally wherein the buffer has a pKa between 7 and 8 and is compatible with Ca2+.

44. The method according to claim 43, wherein the buffer is HNBSACa2+ buffer or BSA buffer.

45. The method according to claim 1, wherein the ratio of TFPI alpha to FXa is approximately 1:1.

46. The method according to claim 1, wherein the concentration of FXa is between 0.1 to 1 nM, optionally wherein the concentration is between 0.2 to 0.6 nM, optionally wherein the concentration is 0.3 nM.

47. The method according to claim 1, wherein the method is performed in conjunction with a method to determine total protein S levels.

48. The method according to claim 1, wherein the method does not comprise a thrombin generation assay.

49. A method of treatment comprising identifying a subject with protein S deficiency using a method according to claim 1, and administering to said subject a therapeutic agent.

50. A method according to claim 49, wherein the therapeutic agent is an anticoagulant therapy.

51. A therapeutic agent for use in treating a subject with a protein S deficiency, wherein the subject has been diagnosed as having a protein S deficiency according to claim 30, optionally wherein the therapeutic agent is an anticoagulant.

52. A kit for determining functional protein S levels in a sample, comprising any two or more of: FV-short (or an FV-short variant; or a functionally-equivalent FV-variant); TFPIα; FXa; phospholipid vesicles; and an FXa substrate, optionally wherein the FXa substrate is S2765.

53. A kit for determining functional protein S levels in sample using a method according to claim 1.


SEQUENCE LISTING (5 sequences)

SEQ ID NO: 1 - protein, 2196 aa, Homo sapiens
AQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDPLQPDVTGIRLLSLGAGEFKSQEHAKHKGPKVERDQAAKHRFSWMKLLAHKVGRHLSQDTGSPSGMRPWEDLPSQDTGSPSRMRPWKDPPSDLLLLKQSNSSKILVGRWHLASEKGSYEIIQDTDEDTAVNNWLISPQNASRAWGESTPLANKPGKQSGHPKFPRVRHKSLQVRQDGGKSRLKKSQFLIKTRKKKKEKHTHHAPLSPRTFHPLRSEAYNTFSERRLKHSLVLHKSNETSLPTDLNQTLPSMDFGWIASLPDHNQNSSNDTGQASCPPGLYQTVPPEEHYQTFPIQDPDQMHSTSDPSHRSSSPELSEMLEYDRSHKSFPTDISQMSPSSEHEVWQTVISPDLSQVTLSPELSQTNLSPDLSHTTLSPELIQRNLSPALGQMPISPDLSHTTLSPDLSHTTLSLDLSQTNLSPELSQTNLSPALGQMPLSPDLSHTTLSLDFSQTNLSPELSHMTLSPELSQTNLSPALGQMPISPDLSHTTLSLDFSQTNLSPELSQTNLSPALGQMPLSPDPSHTTLSLDLSQTNLSPELSQTNLSPDLSEMPLFADLSQIPLTPDLDQMTLSPDLGETDLSPNFGQMSLSPDLSQVTLSPDISDTTLLPDLSQISPPPDLDQIFYPSESSQSLLLQEFNESFPYPDLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 2 - protein, 1494 aa, Homo sapiens
AQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNINLGQMPSPSSPTLNDTFLSKEFNPLVIVGLSKDGTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 3 - protein, 1515 aa, Homo sapiens
AQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGIRSFRNSSLNQEEEEFNLTALALENGTEFVSSNTDIIVGSNYSSPSNISKFTVNNLAEPQKAPSHQQATTAGSPLRHLIGKNSVLNSSTAEHSSPYSEDPIEDTDYIEIIPKEEVQSSEDDYAEIDYVPYDDPYKTDVRTNINSSRDPDNIAAWYLRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPVDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSITLRLELFGCDIY

SEQ ID NO: 4 - protein, 1428 aa, Homo sapiens
AQLRQFYVAAQGISWSYRPEPTNSSLNLSVTSFKKIVYREYEPYFKKEKPQSTISGLLGPTLYAEVGDIIKVHFKNKADKPLSIHPQGIRYSKLSEGASYLDHTFPAEKMDDAVAPGREYTYEWSISEDSGPTHDDPPCLTHIYYSHENLIEDFNSGLIGPLLICKKGTLTEGGTQKTFDKQIVLLFAVFDESKSWSQSSSLMYTVNGYVNGTMPDITVCAHDHISWHLLGMSSGPELFSIHFNGQVLEQNHHKVSAITLVSATSTTANMTVGPEGKWIISSLTPKHLQAGMQAYIDIKNCPKKTRNLKKITREQRRHMKRWEYFIAAEEVIWDYAPVIPANMDKKYRSQHLDNFSNQIGKHYKKVMYTQYEDESFTKHTVNPNMKEDGILGPIIRAQVRDTLKIVFKNMASRPYSIYPHGVTFSPYEDEVNSSFTSGRNNTMIRAVQPGETYTYKWNILEFDEPTENDAQCLTRPYYSDVDIMRDIASGLIGLLLICKSRSLDRRGIQRAADIEQQAVFAVFDENKSWYLEDNINKFCENPDEVKRDDPKFYESNIMSTINGYVPESITTLGFCFDDTVQWHFCSVGTQNEILTIHFTGHSFIYGKRHEDTLTLFPMRGESVTVTMDNVGTWMLTSMNSSPRSKKLRLKFRDVKCIPDDDEDSYEIFEPPESTVMATRKMHDRLEPEDEESDADYDYQNRLAAALGTREFNPLVIVGLSKDGTDYIEIIPKEEVQSSEDDYAEIDYVPYDDPYKTDVRTNINSSRDPDNIAAWYTRSNNGNRRNYYIAAEEISWDYSEFVQRETDIEDSDDIPEDTTYKKVVFRKYLDSTFTKRDPRGEYEEHLGILGPIIRAEVDDVIQVRFKNLASRPYSLHAHGLSYEKSSEGKTYEDDSPEWFKEDNAVQPNSSYTYVWHATERSGPESPGSACRAWAYYSAVNPEKDIHSGLIGPLLICQKGILHKDSNMPMDMREFVLLFMTFDEKKSWYYEKKSRSSWRLTSSEMKKSHEFHAINGMIYSLPGLKMYEQEWVRLHLLNIGGSQDIHVVHFHGQTLLENGNKQHQLGVWPLLPGSFKTLEMKASKPGWWLLNTEVGENQRAGMQTPFLIMDRDCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGCEVNGCSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSIALRLELFGCDIY

SEQ ID NO: 5 - protein, 251 aa, Homo sapiens
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFVTKEGTNDGWKNAAHIYQVFLNAFCIHASMFFLGLDSISCLC